(19) United States
(12) Patent Application Publication
(10) Pub. No.: US 2024/0408073 A1
(43) Pub. Date: Dec. 12, 2024

(54) METHODS FOR TREATING A TRAUMA PATIENT

(71) Applicant: Beth Israel Deaconess Medical Center, Inc., Boston, MA (US)

(72) Inventors: Carl J. Hauser, Boston, MA (US); Leo E. Otterbein, Beverly, MA (US)

(21) Appl. No.: 18/700,227

(22) PCT Filed: Oct. 13, 2022
(86) PCT No.: PCT/US2022/078028

§ 371 Date: Apr. 10, 2024

Related U.S. Application Data
(60) Provisional application No. 63/256,054, filed on Oct. 15, 2021.

(51) Int. Cl. A61K 31/4525; A61K 31/19; A61P 31/04

(52) U.S. Cl. CPC A61K 31/4525; A61K 31/19; A61P 31/04

(57) ABSTRACT

Provided herein are methods of treating a trauma patient, the method including: (a) administering a HDAC inhibitor to the trauma patient; and (b) administering a GRK2 inhibitor to the trauma patient, wherein the treatment prevents, reduces, or ameliorates the risk of an infection occurring after a traumatic event. In some cases, the infection is a respiratory infection, such as pneumonia. The HDAC inhibitor and/or the GRK2 inhibitor can be administered to the patient before, after, or before and after the traumatic event.

### CROSS-REFERENCE TO RELATED APPLICATIONS

This application claims priority to U.S. Provisional Application Ser. No. 63/256,054, filed on Oct. 15, 2021. The disclosure of the prior application is considered part of the disclosure of this application, and is incorporated herein by reference in its entirety.

### FEDERALLY SPONSORED RESEARCH OR DEVELOPMENT

This invention was made with government support under Grant No. W81XWH-16-1-0464, awarded by the United States Army. The government has certain rights to the invention.

### TECHNICAL FIELD

The present disclosure relates to the treatment of trauma patients. In particular, it relates to treatment of health-care associated infections in injured patients.

### BACKGROUND

Trauma is responsible for approximately 8% of all deaths worldwide. Nosocomial infection is the most common cause of morbidity and mortality in patients who survive their initial injury and the observed rates of many types of infections are far higher after injury than would otherwise be expected. The underlying mechanisms linking trauma to nosocomial infection are incompletely defined and improved understanding of those mechanisms should lead to important therapeutic advances.

Effective anti-microbial responses depend upon the coordinated control of leukocyte function. Injury however, creates a “Systemic Inflammatory Response Syndrome” (SIRS) that modifies polymorphonuclear neutrophil (PMN) function and any reduction in PMN antimicrobial function might contribute to susceptibility to infection. Innate immunocytes like PMN are initially activated by “danger signals” in many disease processes. These can be the endogenous danger-associated molecular patterns (DAMPs) released by injury, or the pathogen-associated molecular patterns (PAMPs) that circulate in the setting of sepsis. Danger signals act on a wide variety of immune receptors, including G-protein coupled receptors (GPCR), toll-like receptors (TLRs) and other immune receptors. Such primary signals then give rise to a plethora of secondary signals that include cytokines, chemokines and other mediators of inflammation which can act on immune receptors to localize, amplify or regulate inflammation. Thus depending on the timing, concentration and sequence of agonist exposure, these interactions may be functional and clear infection, or may be dysfunctional by virtue of causing hypofunction, hyperfunction, or spatial maldistribution of responses that can predispose to infection or inflammatory organ injury.

A wide variety of molecular signals mediate PMN anti-microbial function. It has been shown that traumatic injuries can release mitochondrial (mt)DAMPs and circulating mitochondrial formyl peptides (mtFPs) and mtDNA are known to be associated with secondary infection risk after both trauma and primary sepsis. Both bacterial and mitochondrial FPs activate PMN primarily through formyl peptide receptor-1 (FPR1), and PMN exposure to mtFPs decreases their response to bacterial agonists via regulation of multiple GPCRs. Such regulation of receptor expression is due to the activation of G-protein receptor kinases (GRKs) that can internalize GPCRs. The GRKs are a family of seven serine/threonine protein kinases that phosphorylate GPCRs and GRK2 is considered one of the main GRKs regulating PMN function.

Therefore, there remains a need to study the mechanisms by which mtDNA, which can be released in both sterile and infective STRS, might suppress neutrophil chemotaxis and thus potentially make patients with trauma or primary sepsis less able to control secondary infections.

### SUMMARY

Provided herein are methods for treating a trauma patient, comprising: (a) administering a HDAC inhibitor to the trauma patient; and (b) administering a GRK2 inhibitor to the trauma patient. In some embodiments, the HDAC inhibitor and the GRK2 inhibitor are administered at the same time. In some embodiments, the HDAC inhibitor and the GRK2 inhibitor are administered sequentially. In some embodiments, the HDAC inhibitor is administered before the GRK2 inhibitor is administered. In some embodiments, the HDAC inhibitor is administered after the GRK2 inhibitor is administered. In some embodiments, the HDAC inhibitor comprises vorinostat, panobinostat, belinostat, romidepsin, chidamide, valproic acid, tacedinaline, mocetinostat, abexinostat, practinostat, resminostat, givinostat, quisinostat, HBI-8000, or combinations thereof. In some embodiments, the HDAC inhibitor is valproic acid. In some embodiments, the GRK2 inhibitor comprises a selective serotonin reuptake inhibitor, GSK180736A, CMPD101, CMPD103, or combinations thereof. In some embodiments, the GRK2 inhibitor is paroxetine. In some embodiments, the HDAC inhibitor comprises oral administration, intravenous administration, transdermal administration, inhalation administration, or intraosseous vascular administration. In some embodiments, the GRK2 inhibitor comprises oral administration, intravenous administration, transdermal administration, inhalation administration, or intraosseous vascular administration. In some embodiments, the trauma comprises clinical trauma, physical trauma, or combat trauma. In some embodiments, the clinical trauma comprises surgery, injury, tissue damage, infection, inflammation, pain, medical treatment, secondary disease, or combinations thereof. In some embodiments, the infection is a nosocomial infection. In some embodiments, the nosocomial infection is pneumonia. In some embodiments, the nosocomial infection comprises post-injury pneumonia.

Additionally, provided herein are methods of treating a nosocomial infection in a patient, comprising: (a) administering a HDAC inhibitor to the subject; and (b) administering a GRK2 inhibitor to the subject, thereby treating the nosocomial infection in the subject. In some embodiments, the HDAC inhibitor and the GRK2 inhibitor are administered at the same time. In some embodiments, the HDAC inhibitor and the GRK2 inhibitor are administered sequentially.

In some embodiments, the HDAC inhibitor is administered before the GRK2 inhibitor is administered. In some embodiments, the HDAC inhibitor is administered after the GRK2 inhibitor is administered. In some embodiments, the subject has experienced a clinical trauma. In some embodiments, the HDAC inhibitor is administered after the subject experiences the clinical trauma. In some embodiments, the GRK2 inhibitor is administered after the subject experiences the clinical trauma. In some embodiments, the HDAC inhibitor and the GRK2 inhibitor are both administered after the subject experiences the clinical trauma. In some embodiments, the clinical trauma comprises surgery, injury, tissue damage, infection, inflammation, pain, medical treatment, secondary disease, or combinations thereof. In some embodiments, the infection is a nosocomial infection. In some embodiments, the nosocomial infection is pneumonia. In some embodiments, the nosocomial infection comprises post-injury pneumonia. In some embodiments, the HDAC inhibitor comprises vorinostat, panobinostat, belinostat, romidepsin, chidamide, valproic acid, tacedinaline, mocetinostat, abexinostat, practinostat, resminostat, givinostat, quisinostat, HBI-8000, or combinations thereof. In some embodiments, the HDAC inhibitor is valproic acid. In some embodiments, the GRK2 inhibitor comprises a selective serotonin reuptake inhibitor, GSK180736A, CMPD101, CMPD103, or combinations thereof. In some embodiments, the GRK2 inhibitor is a selective serotonin reuptake inhibitor. In some embodiments, the GRK2 inhibitor comprises citalopram, escitalopram, fluoxetine, paroxetine, sertraline, or combinations thereof. In some embodiments, the GRK2 inhibitor is paroxetine. In some embodiments, the HDAC inhibitor comprises oral administration, intravenous administration, transdermal administration, inhalation administration, or intraosseous vascular administration. In some embodiments, the GRK2 inhibitor comprises oral administration, intravenous administration, transdermal administration, inhalation administration, or intraosseous vascular administration.

Additionally, provided herein are methods of prophylactically treating a subject, comprising: (a) administering a HDAC inhibitor to the subject; and (b) administering a GRK2 inhibitor to the subject, wherein the subject is at risk of experiencing trauma. In some embodiments, the HDAC inhibitor and the GRK2 inhibitor are administered at the same time. In some embodiments, the HDAC inhibitor and the GRK2 inhibitor are administered sequentially.

In some embodiments, the HDAC inhibitor is administered before the GRK2 inhibitor is administered. In some embodiments, the HDAC inhibitor is administered after the GRK2 inhibitor is administered. In some embodiments, the HDAC inhibitor is administered before the subject experiences the trauma. In some embodiments, the GRK2 inhibitor is administered before the subject experiences the trauma. In some embodiments, the HDAC inhibitor and the GRK2 inhibitor are both administered before the subject experiences the trauma. In some embodiments, the HDAC inhibitor is administered after the subject experiences the trauma. In some embodiments, the GRK2 inhibitor is administered after the subject experiences the trauma.

In some embodiments, the HDAC inhibitor and the GRK2 inhibitor are both administered after the subject experiences the trauma. In some embodiments, the HDAC inhibitor is administered before and after the subject experiences the trauma. In some embodiments, the GRK2 inhibitor is administered before after the subject experiences the trauma. In some embodiments, the HDAC inhibitor and the GRK2 inhibitor are both administered before and after the subject experiences the trauma. In some embodiments, the HDAC inhibitor comprises vorinostat, panobinostat, belinostat, romidepsin, chidamide, valproic acid, tacedinaline, mocetinostat, abexinostat, practinostat, resminostat, givinostat, quisinostat, HBI-8000, or combinations thereof. In some embodiments, the HDAC inhibitor is valproic acid. In some embodiments, the GRK2 inhibitor comprises a selective serotonin reuptake inhibitor, GSK180736A, CMPD101, CMPD103, or combinations thereof. In some embodiments, the GRK2 inhibitor is a selective serotonin reuptake inhibitor. In some embodiments, the GRK2 inhibitor comprises citalopram, escitalopram, fluoxetine, paroxetine, sertraline, or combinations thereof. In some embodiments, the GRK2 inhibitor is paroxetine. In some embodiments, the HDAC inhibitor comprises oral administration, intravenous administration, transdermal administration, inhalation administration, or intraosseous vascular administration. In some embodiments, the GRK2 inhibitor comprises oral administration, intravenous administration, transdermal administration, inhalation administration, or intraosseous vascular administration. In some embodiments, the trauma comprises surgery, injury, tissue damage, infection, inflammation, pain, medical treatment, secondary disease, or combinations thereof. In some embodiments, the infection is a nosocomial infection. In some embodiments, the infection is pneumonia. In some embodiments, the infection comprises post-injury pneumonia.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-IC are graphs showing that mtDNA suppresses PMN chemotaxis via endosomal TLR9. (FIG. 1A) mtDNA suppresses chemotaxis to multiple GPCR stimuli including formyl peptides (ND6, fMLF) chemokines (GROα) and lipid agonists (LTB₄) in a dose-dependent fashion. (FIG. 1B) The suppressive effect of mtDNA is blocked by chloroquine, showing dependence on endosomal acidification. (FIG. 1C) The suppressive effect of mtDNA is absent in PMN from TLR9 knockout mice.

FIGS. 2A-2C show that mtDNA does not change GCPR expression or receptor bias. (FIG. 2A) mtDNA fails to suppress surface expression of FPR1, BLT1 and CXCR2 at 5 and 15 minutes. At 60 minutes there is actually a slight increase in CXCR2 expression after PMN exposure to mtDNA. All the receptors are regulated by fMLF (third row). (FIG. 2B) Cytosolic calcium ([Ca²⁺]i) responses to fMLF, LTB4, GROα and PAF in Ca²⁺-free and then Ca²⁺-replete media are identical before (black trace) and after (red trace) exposure to mtDNA. (FIG. 2C) Receptor dependent respiratory burst (RB) responses to fMLF and LTB4 are unaffected by mtDNA.

FIGS. 3A-3H show that mtDNA- and FP-induced suppression of CTX depends on GRK2. mtDNA induced suppression of PMN CTX to both (FIG. 3A) GROα and (FIG. 3B) LTB4 were rescued by the GRK2 inhibitor GRKi. Suppression of PMN CTX to LTB4 after exposure to (FIG. 3C) ND6 and suppression of CTX to (FIG. 3D) LTB4 by mtDNA were also both rescued by the GRK2 inhibitor Paroxetine (PAR). (FIG. 3E) Western blots show mtDNA and ND6 each caused both phosphorylation and expression of PMN GRK2. Time courses of GRK2 phosphorylation and expression (line graphs, below) were distinctly different after PMN stimulation via FPR1 (by ND6) versus TLR9 (by mtDNA). (FIG. 3F) Western blot of PMN from healthy volunteers, volunteers undergoing elective surgery, trauma patients who did not get infection (−infection) and trauma patients who did get infections (+infection). A single representative blot is shown, but 115 subjects were studied. Activation of GRK2 (to pGRK2) was universal in trauma but greatest in patients who developed infection. Reciprocal suppression of cortactin (by acetylation) was also seen especially in patients who developed infection. Active cortactin is required for cytoskeletal elongation and branching. (FIG. 3G) PMN from volunteer controls (VC) showed stable, low baseline levels of GRK2 phosphorylation. PMN from trauma patients destined to develop infections showed 2 to 3-fold enhancement of GRK2 activation. This enhancement was maximal 1 to 3d after injury. *p<0.01, **p<0.001. (FIG. 3H) shows mtDNA impairs acetylation of cortactin, and that paroxetine restores acetylated-cortactin levels. Left panel shows by Western blot that mtDNA decreases the amount of acetylated cortactin (ac-cortactin), and that administration paroxetine (PAR) in conjunction with mtDNA blocks mtDNA suppression of ac-cortactin. Right panel shows the ratio of ac-cortactin to cortactin, for each of the four conditions examined, normalized to the no PAR, no mtDNA (control) condition.

FIGS. 4A-4C show that mtDNA decreases PMN bacterial phagocytosis. (FIG. 4A) Phagocytosis of SYTO9 labeled Staphylococcus Aureus (SA) by human PMN (CD16⁺ on flow cytometry) is suppressed by mtDNA. (FIG. 4B) PMN uptake in co-culture of Sα (PMN lysis/agar plate) and (FIG. 4C) clearance of Sα from co-culture media are suppressed by mtDNA. In all cases, suppression is reversed by either PAR (left) or VPA (right).

FIG. 5 shows that inhibition of F-actin polymerization by mtDNA is rescued by PAR and VPA. Both chemotaxis and phagocytosis depend on G-actin polymerization to F-actin filaments. It is shown here that LTB4 causes brisk polymerization (Upper 3^{rd} panel) compared to control (Lt upper panel). Polymerization responses to LTB4 are lost in the presence of mtDNA (Upper Rt panel) but rescued by the presence of PAR (Lower 2^{nd} panel) or VPA (Lower Rt panel). No significant changes were noted in the absence of LTB4. (Phalloidin stain, ×200, scale bar=50 m).

FIGS. 6A-6C show in-vivo effects of single vs dual GRK pathway inhibition on infection after trauma. Based on preceding delineation of the effects of injury-derived DAMPs on PMN GRK signaling and related cellular functions, it was studied whether GRK inhibition might reverse the suppression of antimicrobial function by injury seen in-vivo. Control (Uninjured) CD-1 mice clear bacteria (1.8×10⁸Sα injected intra-tracheal) overnight. Animals undergoing Trauma (laparotomy+liver crush) 4 hours before Sa injection fail to clear the inoculum. (FIG. 6A) Pre-treatment with PAR (20 mg/kg, 30 min prior to injury) significantly prevented infection. Trauma is unpredictable, however, so subsequent to this finding post-treatments were studied. (FIG. 6B) Given post-injury however, it was found that single-drug therapies (PAR 20 mg/kg or VPA 80 mg/kg) did not confer significant protection. In contrast, PAR+VPA given in combination (same doses) after trauma+infection rescued lung bacterial clearance to uninjured levels. n=13 for Uninjured and Trauma groups. N=6-10 for treatment groups. CFU denotes colony forming units of Sa retrieved by BAL and presented as % of uninjured controls. (FIG. 6C) Mice treated as in FIG. 6B (here, n=10/group) were observed out to 7 days for survival. Kaplan-Meier survival curves are shown. Treatment with VAL+PAR (given i.p. on days 0, 1 and 2) significantly decreased mortality (p<0.005, Log-rank test.) FIG. 7 is an exemplary schematic showing an overview of how mitochondrial DAMPs released by trauma act on the PMN G-protein coupled receptor kinase (GRK) system. mtDAMPs derived from injury or inflammation can affect neutrophil GRKs either by direct interaction of mtFPs with cell-surface formyl peptide receptors (FPRs), or by mtDNA interactions with TLR9. FPRs activate GRKs through a canonical pathway that internalizes GPCRs. mtDNA activates GRKs via TLR9, which activates a novel non-canonical pathway that can phosphorylate HDAC6 and so interfere with cytoskeletal assembly. PAR prevents GRK activation. VPA acts on HDACs downstream from activated GRK2 to rescue impaired cytoskeletal reorganization.

FIGS. 8A-B is a set of bar graphs where PMN from traumatized mice and humans show decreased Toll-like-Receptor-2 (TLR2) expression, indicating that trauma increases susceptibility to infection in mice and humans. FIG. 8A shows the effect of trauma on mouse lung bacterial clearance, where results show TLR2 on PMN (blood and lung/BAL) is markedly reduced after trauma. FIG. 8B shows identical effects of trauma on human PMN TLR2. CD16 is also decreased, but CD66b is unchanged, showing specificity. Finally, mouse PMN exposed to trauma plasma generate reactive oxygen species (ROS), which hampers PMN's ability to kill bacterial cells.

FIGS. 9A-9G show that plasma from trauma patients suppress PMN function. (FIG. 9A) Respiratory burst is immediately suppressed (FIG. 9B), but the suppressive effect declines over 2-3 days. (FIG. 9C) Mitochondrial (mt) DNA suppresses PMN chemotaxis to the agonists fMLF and mitochondrial formyl peptide (ND6). (FIG. 9D) Trauma patients' PMN show activation and increased expression of GRK2. (FIG. 9E) PMN respiratory burst (RB) is suppressed by plasma from trauma patients (TP) but not control patients (CP). Pre-treatment with PAR can rescue RB but VAL and PAR alone are ineffective as post-treatments. VAL and PAR together (VAL+PAR) rescue respiratory burst post-treatment. (FIG. 9F) Neutrophil extracellular trap formation (NETosis): PMN were incubated with volunteer or trauma plasma. NETosis was then induced by phorbol myristate acetate (PMA). Trauma markedly suppresses NET formation. Post-treatment (30 min after plasma) shows minor reversals by VAL or PAR alone, but VAL+PAR significantly rescued NETosis. (FIG. 9G) Receptor expression: human PMN are stained for surface expression of Formyl Peptide Receptor-1 (FPR1). In the upper panel, the mtDAMP ND6 is seen to suppress FPR1 expression. In the lower panel, VAL and PAR each diminish FPR1 regulation by ND6. The effect is additive, and together VAL+PAR return FPR1 expression to completely normal. Paroxetine is highly selective for GRK2, having a Kd between 0.1 and 1 M both in published animal studies and in our hands in human PMN (not shown).

FIG. 10 is a bar graph showing that GRK2 is phosphorylated in human PMN incubated in trauma plasma as opposed to control plasma.

FIG. 11 is a bar graph showing results from agar plate assays of supernatants from PMN incubated with Staph. Aureus in the presence of 10% control plasma, 10% trauma plasma or 10% trauma plasma+VAL+PAR. N=6 plasma samples/group. The suppression of PMN bacterial killing by trauma plasma is reversed by VAL+PAR.

FIG. 12 is an exemplary schematic showing an overview of how injury activates G-protein coupled receptor (GPCR) kinases (GRKs) and suppresses PMN function. Left: Normally PMN sense infection via chemoattractants (CTX) and GPCRs, then kill the bacteria using effector functions like 02-burst. Center: After injury, DAMPs and inflammatory mediators affect PMN by interacting with GPC receptors (for FPs, chemokines, leukotrienes, complement) or via TLRs. TLR9 interacts with DAMPs like mtDNA. GPC receptors activate canonical GRK pathways (blue arrows) where β-arrestin internalizes multiple GPCRs. mtDNA binds TLR9 to activate a novel non-canonical pathway (green arrows) that interferes with cytoskeletal function. It was found that both GRK pathways suppress a range of PMN antimicrobial effector functions. Right: PAR inhibits GRK2, preventing canonical effects. VAL acts on HDAC to rescue effector functions dependent on cytoskeletal organization.

FIGS. 13A-13C show effects of therapeutic VAL+PAR on trauma-induced susceptibility to lung infection in the pig. (FIG. 13A) Gross images of lungs from pigs subjected to liver trauma followed by S. aureus (10¹⁰ cfu) inoculated into the right cranial (upper) and lower lobes. Both specimens received liver injury plus bacteria. The right specimen shows the beneficial effects of VAL+PAR. (FIG. 13B) Sα CFU counts in BAL done 24 h after lung inoculation in uninjured controls, liver injured pigs and liver-injured pigs treated with VAL (10 mg/kg, i.v.) and PAR (1 mg/kg, p.o.). Animals with liver injury 48h before bacteria showed poor bacterial clearance compared to control. VAL+PAR treatment rescued this by ˜50%. (FIG. 13C) Bacterial counts in right lung tissue 24h post inoculation with S. aureus. The combination of VAL+PAR rescued trauma-induced deficits in bacterial clearance. CFR results in parentheses represent mean±SD of 4 animals/group. * p<0.001.

### DETAILED DESCRIPTION

Trauma (e.g., injury, surgery, tissue damage, or secondary disease) predisposes a subject to infection wherein leukocytes, like polymorphonuclear neutrophils (PMN) for example, are critical for pathogen control. Mitochondrial (mt)DAMPs, such as mtDNA and formyl peptides (mtFP) can be released by trauma or inflammation, and are associated with infection risk. It has been shown that FPR-1 activation by mtFPs internalizes G-protein coupled receptors (GPCR) and globally suppresses chemotaxis (CTX). mtDNA suppressive actions require toll-like receptor 9 (TLR9), and mtDNA and mtFPs both activate GRK2 but act by different pathways, wherein mtDNA suppression of PMN CTX is rescued by GRK2 inhibitors like paroxetine, but this GRK activation is not ‘canonical’ since GPCR expression and bias were unchanged. Rather, mtDNA impaired F-actin assembly, suggesting histone deacetylase (HDAC)-mediated disruption of actin polymerization. mtDNA stimulation of non-canonical GRK activity can be further supported in that PMN F-actin formation, CTX, bacterial phagocytosis and killing in the presence of mtDNA were all rescued by the HDAC inhibitor valproic acid. The importance of these dual pathways is demonstrated in-vivo where traumatic suppression of pulmonary bacterial clearance is rescued by combining GRK and HDAC inhibition.

Provided herein are methods for treating a trauma patient, the method including: (a) administering a HDAC inhibitor to the trauma patient; and (b) administering a GRK2 inhibitor to the trauma patient.

Also provided herein are methods of treating a nosocomial infection in a patient, the method including: (a) administering a HDAC inhibitor to the subject; and (b) administering a GRK2 inhibitor to the subject, thereby treating the nosocomial infection in the patient.

Also provided herein are methods of prophylactically treating a subject, the method including: (a) administering a HDAC inhibitor to the subject; and (b) administering a GRK2 inhibitor to the subject, wherein the subject is at risk of experiencing a clinical trauma.

Various non-limiting aspects of these methods are described herein, and can be used in any combination without limitation. Additional aspects of various components of methods for treating a trauma patient, treating a nosocomial infection in a subject, or prophylactically treating a subject are known in the art.

It must be noted that, as used in the specification and the appended claims, the singular forms “a,” “an” and “the” include plural referents unless the context clearly dictates otherwise.

As used herein, the term “administration” can refer to the administration of a composition to a subject or system to achieve delivery of the composition. Those of ordinary skill in the art will be aware of a variety of routes that may, in appropriate circumstances, be utilized for administration to a subject, for example a human. For example, in some embodiments, administration may be ocular, oral, parenteral, or topical. In some embodiments, administration may be bronchial (e.g., by bronchial instillation), buccal, dermal (which may be or comprise, for example, one or more of topical to the dermis, intradermal, interdermal, or transdermal), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, or vitreal. In some embodiments, administration may involve only a single dose. In some embodiments, administration may involve administration of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the terms “effective amount” and “effective to treat” can refer to an amount of concentration of a HDAC inhibitor and/or a GRK2 inhibitor utilized for a period of time (e.g., acute or chronic administration, periodic or continuous administration) that is effective within the context of its administration for causing an intended effect or physiological outcome. In some embodiments, an effective amount of a HDAC inhibitor and/or a GRK2 inhibitor can be an amount that reduces susceptibility to infection associated with trauma (e.g., tissue injury, surgery). In some embodiments, an effective amount of a HDAC inhibitor and/or a GRK2 inhibitor can be an amount that reverses leukocyte dysfunction (e.g., leukocyte chemotactic dysfunction, leukocyte phagocytic dysfunction). In some embodiments, an effective amount of a HDAC inhibitor and/or a GRK2 inhibitor can be an amount that treats a nosocomial infection. In some embodiments, an effective amount of a HDAC inhibitor and/or a GRK2 inhibitor can be an amount that prophylactically treats a subject that is at risk of experiencing a clinical trauma. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen. Further, the dose to be administered can vary depending upon the age, weight, and general condition of the patient as well as the severity of the condition being treated, the judgment of the healthcare professional, and the particular mode of administration.

As used herein, the terms “subject” and “patient” are used interchangeably throughout the specification to describe an organism, typically a mammal, human or non-human, to whom treatment according to the methods of the present disclosure is provided. Veterinary applications are contemplated by the present disclosure. The terms include, but are not limited to, mammals, e.g., humans, other primates, pigs, hamsters, mice, rats, cows, horses, cats, dogs, sheep, and goats. In some embodiments, a subject is suffering from a relevant disease, disorder, or condition. In some embodiments, a subject is a trauma patient. In some embodiments, a subject is susceptible to a disease, disorder, or condition. In some embodiments, a subject is at risk of experiencing a trauma. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, a “trauma” can refer to an incident or other traumatic event that causes physical harm. In some embodiments the trauma can be clinical trauma, physical trauma, or combat trauma. In some embodiments, a trauma can include a clinical trauma (e.g., injury, infection, secondary disease, medical procedures, surgery, inflammation, tissue damage, medical treatment, or combinations thereof). In some embodiments, the clinical trauma occurs within the context of a medical or other healthcare setting, such as a surgery or other medical procedure. In some embodiments, the clinical trauma occurs outside the context of a medical or other healthcare setting, such as a patient's chronic disease or condition. In some embodiments, a trauma is typically in the form of a physical injury, wherein external force or energy is applied to the subject. In some embodiments, a trauma can include an injury (e.g., a wound) to living tissue caused by an extrinsic agent. In some embodiments, a trauma can include blunt force trauma or a penetrating trauma. In some embodiments, a trauma can include an accident, injury, or an attack that was unexpected or sudden. In some embodiments, the physical trauma is a combat trauma. A combat trauma is a trauma that occurs in the context of or the result of engaging in military fighting. In some embodiments, a combat trauma can include blast injury, burn injury, or hemorrhagic shock.

Skilled practitioners will appreciate that a patient can be diagnosed by a physician as suffering from or at risk of experiencing a trauma (e.g., clinical trauma, physical trauma, or combat trauma). Subjects considered at risk for experiencing trauma may benefit particularly from the methods in present disclosure, particularly because prophylactic treatment can begin before the subject experiencing any type of trauma. Individuals “at risk” include, e.g., subjects exposed to environmental, occupational, therapeutic elements that may cause trauma. In some embodiments, a trauma includes a clinical trauma, a physical trauma, or a combat trauma. The skilled practitioner will appreciate that a patient can be determined to be at risk of experiencing a trauma by medical personnel evaluation. In some embodiments, the subject at risk of experiencing trauma does not require medical personnel evaluation, but the risk is assumed by activity or occupational hazard (e.g., military personnel). In some embodiments, a trauma cannot be predicted. In some embodiments, a patient can be assumed to be at risk of experiencing a trauma by activity or occupational hazard but the risk cannot be determined to rise to the level of indicating medical treatment.

The HDAC and/or GRK2 inhibitors described herein may be used to treat, or prophylactically treat the risk of, post-injury infection. The infection can be a respiratory infection, local infection, or systemic infection. The infection can be a bacterial infection. The infection can be a nosocomial infection. The infection can be caused by Streptococcus bacteria, such as Streptococcus pneumoniae, Staphylococcus aureus, and Group A Streptococcus. The infection can be caused by other types of bacteria, such as Klebsiella pneumoniae, Haemophilus influenzae, Moraxella catarrhalis, or P. aeruginosa. In some embodiments, the infection can be caused by Gram-negative bacteria. In some embodiments, the infection can be caused by Gram-positive bacteria. In some embodiments, the infection can be caused by Gram-negative bacteria or Gram-positive bacteria. The bacteria can cause pneumonia. The pneumonia can be nosocomial pneumonia. The pneumonia can be post-injury pneumonia.

### HDAC Inhibitors

Histone deacetylase (HDAC) inhibitors are chemical compounds that inhibit histone deacetylases. Histones are major protein components of chromatin and the regulation of chromatin structure is emerging as a central mechanism for the control of gene expression. As a general paradigm, acetylation of the ∈-amino groups of lysine residues in the amino-terminal tails of nucleosomal histones is associated with transcriptional activation, while deacetylation is associated with condensation of chromatin and transcriptional repression. Acetylation and deacetylation of histones is controlled by the enzymatic activity of histone acetyltransferases (HATs) and histone deacetylases (HDACs). HDAC inhibitors can induce different phenotypes in various transformed cells, including growth arrest, activation of the extrinsic and/or intrinsic apoptotic pathways, autophagic cell death, mitotic cell death, and senescence. However, in some embodiments, HDAC inhibitors can also have immunomodulatory activity and possess suppressive effects on immune response gene induction. HDAC inhibitors have been used in psychiatry and neurology as mood stabilizers and anti-epileptics.

Typically, HDAC inhibitors can include hydroxamic acid derivatives, Short-Chain Fatty Acids (SCFAs), cyclic tetrapeptides, benzamides, electrophilic ketones, and/or any other class of compounds capable of inhibiting histone deacetylases. Non-limiting examples of such HDAC inhibitors can include, but are not limited to, suberoylanilide hydroxamic acid (SAHA), m-carboxycinnamic acid bishydroxamide (CBHA), pyroxamide, trichostatin A (TSA), trichostatin C, salicylhydroxamic acid, suberoyl bishydroxamic acid (SBHA), azelaic bishydroxamic acid (ABHA), PXD-101 (Prolifix); LAQ-824; CHAP; MW2796, or MW2996. In some embodiments, a HDAC inhibitor can be vorinostat, panobinostat, belinostat, romidepsin, chidamide, valproic acid, tacedinaline, mocetinostat, abexinostat, practinostat, resminostat, givinostat, quisinostat, HBI-8000, or combinations thereof. In some embodiments, a HDAC inhibitor can be valproic acid.

In some embodiments, a HDAC inhibitor can be administered to a subject in a therapeutically effective amount. In some embodiments, a HDAC inhibitor can be administered alone or as part of a pharmaceutically acceptable composition or formulation. In some embodiments, a HDAC inhibitor can be administered in combination with one or more additional pharmaceutically active compounds. In some embodiments, a HDAC inhibitor can be administered all at once, multiple or divided administrations, or delivered over a period of time. In some embodiments, a HDAC inhibitor can be administered to a patient or subject by any suitable route, e.g., orally, rectally, intravenously, intramuscularly, subcutaneously, intracisternally, intravaginally, intraperitoneally, intravesically, or as a buccal, inhalation, or nasal spray. In some embodiments, the HDAC inhibitor can be administered to a patient through oral administration, intravenous administration, transdermal administration, inhalation administration, or intraosseous vascular administration. In some embodiments, the dosage of the HDAC inhibitor to be administered can be varied over time.

Therapeutic amounts can be empirically determined by those of skill in the art, and can vary with the condition being treated, the subject, and the efficacy and toxicity of each of the active agents contained in the composition. In some embodiments, a preferred range of a dosage of a HDAC inhibitor can be about 0.01 mg/kg to about 100 mg/kg (e.g., about 0.01 mg/kg to about 90 mg/kg, about 0.01 mg/kg to about 80 mg/kg, about 0.01 mg/kg to about 70 mg/kg, about 0.01 mg/kg to about 60 mg/kg, about 0.01 mg/kg to about 50 mg/kg, about 0.01 mg/kg to about 40 mg/kg, about 0.01 mg/kg to about 30 mg/kg, about 0.01 mg/kg to about 20 mg/kg, about 0.01 mg/kg to about 10 mg/kg, about 0.01 mg/kg to about 5 mg/kg, about 0.01 mg/kg to about 1 mg/kg, about 0.01 mg/kg to about 0.5 mg/kg, about 0.01 mg/kg to about 0.1 mg/kg, about 0.01 mg/kg to about 0.05 mg/kg, about 0.05 mg/kg to about 100 mg/kg, about 0.05 mg/kg to about 90 mg/kg, about 0.05 mg/kg to about 80 mg/kg, about 0.05 mg/kg to about 70 mg/kg, about 0.05 mg/kg to about 60 mg/kg, about 0.05 mg/kg to about 50 mg/kg, about 0.05 mg/kg to about 40 mg/kg, about 0.05 mg/kg to about 30 mg/kg, about 0.05 mg/kg to about 20 mg/kg, about 0.05 mg/kg to about 10 mg/kg, about 0.05 mg/kg to about 5 mg/kg, about 0.05 mg/kg to about 1 mg/kg, about 0.05 mg/kg to about 0.5 mg/kg, about 0.05 mg/kg to about 0.1 mg/kg, about 0.1 mg/kg to about 100 mg/kg, about 0.1 mg/kg to about 90 mg/kg, about 0.1 mg/kg to about 80 mg/kg, about 0.1 mg/kg to about 70 mg/kg, about 0.1 mg/kg to about 60 mg/kg, about 0.1 mg/kg to about 50 mg/kg, about 0.1 mg/kg to about 40 mg/kg, about 0.1 mg/kg to about 30 mg/kg, about 0.1 mg/kg to about 20 mg/kg, about 0.1 mg/kg to about 10 mg/kg, about 0.1 mg/kg to about 5 mg/kg, about 0.1 mg/kg to about 1 mg/kg, about 0.1 mg/kg to about 0.5 mg/kg, about 0.5 mg/kg to about 100 mg/kg, about 0.5 mg/kg to about 90 mg/kg, about 0.5 mg/kg to about 80 mg/kg, about 0.5 mg/kg to about 70 mg/kg, about 0.5 mg/kg to about 60 mg/kg, about 0.5 mg/kg to about 50 mg/kg, about 0.5 mg/kg to about 40 mg/kg, about 0.5 mg/kg to about 30 mg/kg, about 0.5 mg/kg to about 20 mg/kg, about 0.5 mg/kg to about 10 mg/kg, about 0.5 mg/kg to about 5 mg/kg, about 0.5 mg/kg to about 1 mg/kg, about 1 mg/kg to about 100 mg/kg, about 1 mg/kg to about 90 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 1 mg/kg to about 5 mg/kg, about 5 mg/kg to about 100 mg/kg, about 5 mg/kg to about 90 mg/kg, about 5 mg/kg to about 80 mg/kg, about 5 mg/kg to about 70 mg/kg, about 5 mg/kg to about 60 mg/kg, about 5 mg/kg to about 50 mg/kg, about 5 mg/kg to about 40 mg/kg, about 5 mg/kg to about 30 mg/kg, about 5 mg/kg to about 20 mg/kg, about 5 mg/kg to about 10 mg/kg, about 10 mg/kg to about 100 mg/kg, about 10 mg/kg to about 90 mg/kg, about 10 mg/kg to about 80 mg/kg, about 10 mg/kg to about 70 mg/kg, about 10 mg/kg to about 60 mg/kg, about 10 mg/kg to about 50 mg/kg, about 10 mg/kg to about 40 mg/kg, about 10 mg/kg to about 30 mg/kg, about 10 mg/kg to about 20 mg/kg, about 20 mg/kg to about 100 mg/kg, about 20 mg/kg to about 90 mg/kg, about 20 mg/kg to about 80 mg/kg, about 20 mg/kg to about 70 mg/kg, about 20 mg/kg to about 60 mg/kg, about 20 mg/kg to about 50 mg/kg, about 20 mg/kg to about 40 mg/kg, about 20 mg/kg to about 30 mg/kg, about 30 mg/kg to about 100 mg/kg, about 30 mg/kg to about 90 mg/kg, about 30 mg/kg to about 80 mg/kg, about 30 mg/kg to about 70 mg/kg, about 30 mg/kg to about 60 mg/kg, about 30 mg/kg to about 50 mg/kg, about 30 mg/kg to about 40 mg/kg, about 40 mg/kg to about 100 mg/kg, about 40 mg/kg to about 90 mg/kg, about 40 mg/kg to about 80 mg/kg, about 40 mg/kg to about 70 mg/kg, about 40 mg/kg to about 60 mg/kg, about 40 mg/kg to about 50 mg/kg, about 50 mg/kg to about 100 mg/kg, about 50 mg/kg to about 90 mg/kg, about 50 mg/kg to about 80 mg/kg, about 50 mg/kg to about 70 mg/kg, about 50 mg/kg to about 60 mg/kg, about 60 mg/kg to about 100 mg/kg, about 60 mg/kg to about 90 mg/kg, about 60 mg/kg to about 80 mg/kg, about 60 mg/kg to about 70 mg/kg, about 70 mg/kg to about 100 mg/kg, about 70 mg/kg to about 90 mg/kg, about 70 mg/kg to about 80 mg/kg, about 80 mg/kg to about 100 mg/kg, about 80 mg/kg to about 90 mg/kg, or about 90 mg/kg to about 100 mg/kg).

In some embodiments, the amount of the HDAC inhibitor can be about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, about 100 mg/kg.

### GRK2 Inhibitors

G protein-coupled receptor kinase 2 (GRK2) inhibitors are small molecules to inhibit GRK2, typically for the treatment of heart disease and hypertension. GRKs can be classified in one of three subfamilies based on gene structure and homology. Among the three subfamilies, the GRK2 subfamily, which includes GRK2 and GRK3, are Gβγ-dependent and play important roles in the heart and olfactory neurons, respectively. In particular, GRK2 phosphorylates activated β-adrenergic receptors, thereby preventing overstimulation of cAMP-dependent signaling. Because GRK2 overexpression in the heart is a biomarker for heart failure, inhibitors of GRK2 have been developed for the treatment of cardiovascular disease.

GRK2 inhibitors can include, but are not limited to, balanol, Takeda inhibitors, paroxetine and derivatives, M119 and gallein, peptides, RNA aptamers, RKIP, and microRNAs (miRNAs), which have different structures, inhibition effects, and inhibition mechanisms. In some embodiments, a GRK2 inhibitor can include a selective serotonin reuptake inhibitor, GSK180736A, CMPD101, CMPD103, or combinations thereof. In some embodiments, a GRK2 inhibitor can be a selective serotonin reuptake inhibitor. In some embodiments, a GRK2 inhibitor can include citalopram, escitalopram, fluoxetine, paroxetine, sertraline, or combinations thereof. In some embodiments, a GRK2 inhibitor can be paroxetine.

In some embodiments, a GRK2 inhibitor can be administered to a subject in a therapeutically effective amount. In some embodiments, a GRK2 inhibitor can be administered alone or as part of a pharmaceutically acceptable composition or formulation. In some embodiments, a GRK2 inhibitor can be administered in combination with one or more additional pharmaceutically active compound. In some embodiments, a GRK2 inhibitor can be administered all at once, multiple times, or delivered over a period of time. In some embodiments, a GRK2 inhibitor can be administered to a patient or subject by any suitable route, e.g., orally, rectally, intravenously, intramuscularly, subcutaneously, intracisternally, intravaginally, intraperitoneally, intravesically, or as a buccal, inhalation, or nasal spray. In some embodiments, the GRK2 inhibitor can be administered to a patient through oral administration, intravenous administration, transdermal administration, inhalation administration, or intraosseous vascular administration. In some embodiments, the dosage of the GRK2 inhibitor to be administered can be varied over time.

Therapeutic amounts can be empirically determined by those of skill in the art, and can vary with the condition being treated, the subject, and the efficacy and toxicity of each of the active agents contained in the composition. In some embodiments, a preferred range of a dosage of a GRK2 inhibitor can be about 0.01 mg/kg to about 100 mg/kg (e.g., about 0.01 mg/kg to about 90 mg/kg, about 0.01 mg/kg to about 80 mg/kg, about 0.01 mg/kg to about 70 mg/kg, about 0.01 mg/kg to about 60 mg/kg, about 0.01 mg/kg to about 50 mg/kg, about 0.01 mg/kg to about 40 mg/kg, about 0.01 mg/kg to about 30 mg/kg, about 0.01 mg/kg to about 20 mg/kg, about 0.01 mg/kg to about 10 mg/kg, about 0.01 mg/kg to about 5 mg/kg, about 0.01 mg/kg to about 1 mg/kg, about 0.01 mg/kg to about 0.5 mg/kg, about 0.01 mg/kg to about 0.1 mg/kg, about 0.01 mg/kg to about 0.05 mg/kg, about 0.05 mg/kg to about 100 mg/kg, about 0.05 mg/kg to about 90 mg/kg, about 0.05 mg/kg to about 80 mg/kg, about 0.05 mg/kg to about 70 mg/kg, about 0.05 mg/kg to about 60 mg/kg, about 0.05 mg/kg to about 50 mg/kg, about 0.05 mg/kg to about 40 mg/kg, about 0.05 mg/kg to about 30 mg/kg, about 0.05 mg/kg to about 20 mg/kg, about 0.05 mg/kg to about 10 mg/kg, about 0.05 mg/kg to about 5 mg/kg, about 0.05 mg/kg to about 1 mg/kg, about 0.05 mg/kg to about 0.5 mg/kg, about 0.05 mg/kg to about 0.1 mg/kg, about 0.1 mg/kg to about 100 mg/kg, about 0.1 mg/kg to about 90 mg/kg, about 0.1 mg/kg to about 80 mg/kg, about 0.1 mg/kg to about 70 mg/kg, about 0.1 mg/kg to about 60 mg/kg, about 0.1 mg/kg to about 50 mg/kg, about 0.1 mg/kg to about 40 mg/kg, about 0.1 mg/kg to about 30 mg/kg, about 0.1 mg/kg to about 20 mg/kg, about 0.1 mg/kg to about 10 mg/kg, about 0.1 mg/kg to about 5 mg/kg, about 0.1 mg/kg to about 1 mg/kg, about 0.1 mg/kg to about 0.5 mg/kg, about 0.5 mg/kg to about 100 mg/kg, about 0.5 mg/kg to about 90 mg/kg, about 0.5 mg/kg to about 80 mg/kg, about 0.5 mg/kg to about 70 mg/kg, about 0.5 mg/kg to about 60 mg/kg, about 0.5 mg/kg to about 50 mg/kg, about 0.5 mg/kg to about 40 mg/kg, about 0.5 mg/kg to about 30 mg/kg, about 0.5 mg/kg to about 20 mg/kg, about 0.5 mg/kg to about 10 mg/kg, about 0.5 mg/kg to about 5 mg/kg, about 0.5 mg/kg to about 1 mg/kg, about 1 mg/kg to about 100 mg/kg, about 1 mg/kg to about 90 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 1 mg/kg to about 5 mg/kg, about 5 mg/kg to about 100 mg/kg, about 5 mg/kg to about 90 mg/kg, about 5 mg/kg to about 80 mg/kg, about 5 mg/kg to about 70 mg/kg, about 5 mg/kg to about 60 mg/kg, about 5 mg/kg to about 50 mg/kg, about 5 mg/kg to about 40 mg/kg, about 5 mg/kg to about 30 mg/kg, about 5 mg/kg to about 20 mg/kg, about 5 mg/kg to about 10 mg/kg, about 10 mg/kg to about 100 mg/kg, about 10 mg/kg to about 90 mg/kg, about 10 mg/kg to about 80 mg/kg, about 10 mg/kg to about 70 mg/kg, about 10 mg/kg to about 60 mg/kg, about 10 mg/kg to about 50 mg/kg, about 10 mg/kg to about 40 mg/kg, about 10 mg/kg to about 30 mg/kg, about 10 mg/kg to about 20 mg/kg, about 20 mg/kg to about 100 mg/kg, about 20 mg/kg to about 90 mg/kg, about 20 mg/kg to about 80 mg/kg, about 20 mg/kg to about 70 mg/kg, about 20 mg/kg to about 60 mg/kg, about 20 mg/kg to about 50 mg/kg, about 20 mg/kg to about 40 mg/kg, about 20 mg/kg to about 30 mg/kg, about 30 mg/kg to about 100 mg/kg, about 30 mg/kg to about 90 mg/kg, about 30 mg/kg to about 80 mg/kg, about 30 mg/kg to about 70 mg/kg, about 30 mg/kg to about 60 mg/kg, about 30 mg/kg to about 50 mg/kg, about 30 mg/kg to about 40 mg/kg, about 40 mg/kg to about 100 mg/kg, about 40 mg/kg to about 90 mg/kg, about 40 mg/kg to about 80 mg/kg, about 40 mg/kg to about 70 mg/kg, about 40 mg/kg to about 60 mg/kg, about 40 mg/kg to about 50 mg/kg, about 50 mg/kg to about 100 mg/kg, about 50 mg/kg to about 90 mg/kg, about 50 mg/kg to about 80 mg/kg, about 50 mg/kg to about 70 mg/kg, about 50 mg/kg to about 60 mg/kg, about 60 mg/kg to about 100 mg/kg, about 60 mg/kg to about 90 mg/kg, about 60 mg/kg to about 80 mg/kg, about 60 mg/kg to about 70 mg/kg, about 70 mg/kg to about 100 mg/kg, about 70 mg/kg to about 90 mg/kg, about 70 mg/kg to about 80 mg/kg, about 80 mg/kg to about 100 mg/kg, about 80 mg/kg to about 90 mg/kg, or about 90 mg/kg to about 100 mg/kg).

In some embodiments, the amount of the GRK2 inhibitor can be 0.01 mg/kg, about 0.02 mg/kg, about 00.3 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg.

### Method of Treating a Trauma Patient

Provided herein are methods of treating a trauma patient, the method including: (a) administering a HDAC inhibitor to the trauma patient; and (b) administering a GRK2 inhibitor to the trauma patient. In some embodiments, the HDAC inhibitor and the GRK2 inhibitor are administered at the same time. In some embodiments, the HDAC inhibitor and the GRK2 inhibitor are administered sequentially. In some embodiments, the HDAC inhibitor is administered before the GRK2 inhibitor is administered. In some embodiments, the HDAC inhibitor is administered after the GRK2 inhibitor is administered.

In some embodiments, a trauma patient experiences a trauma including clinical trauma, physical trauma, or combat trauma. In some embodiments, the clinical trauma comprises surgery, injury, tissue damage, infection, inflammation, pain, medical treatment, secondary disease, or combinations thereof. In some embodiments, the infection is a nosocomial infection. In some embodiments, the nosocomial infection is pneumonia. In some embodiments, the nosocomial infection comprises post-injury pneumonia.

Also provided herein are methods of treating a nosocomial infection in a subject, the method including: (a) administering a HDAC inhibitor to the subject; and (b) administering a GRK2 inhibitor to the subject, thereby treating the nosocomial infection in the subject. In some embodiments, the HDAC inhibitor and the GRK2 inhibitor are administered at the same time. In some embodiments, the HDAC inhibitor and the GRK2 inhibitor are administered sequentially. In some embodiments, the HDAC inhibitor is administered before the GRK2 inhibitor is administered. In some embodiments, the HDAC inhibitor is administered after the GRK2 inhibitor is administered. In some embodiments, the subject has experienced a clinical trauma. In some embodiments, the HDAC inhibitor is administered after the subject experiences the clinical trauma. In some embodiments, the GRK2 inhibitor is administered after the subject experiences the clinical trauma. In some embodiments, the HDAC inhibitor and the GRK2 inhibitor are both administered after the subject experiences the clinical trauma.

In some embodiments, the clinical trauma comprises surgery, injury, tissue damage, infection, inflammation, pain, medical treatment, secondary disease, or combinations thereof. In some embodiments, the infection is a nosocomial infection. In some embodiments, the nosocomial infection is pneumonia. In some embodiments, the nosocomial infection comprises post-injury pneumonia.

In some embodiments, the HDAC and/or GRK2 inhibitors are administered to the trauma patient after the trauma event occurred. For example, the HDAC and/or GRK2 inhibitors are administered to the trauma patient within 30 minutes of the subject experiencing the trauma/traumatic event. In other examples, the HDAC and/or GRK2 inhibitors are administered to the trauma patient within 35, 40, 45, 50, 55, 60, 65, 70, 75, 80, 85, 90, 95, 100, 105, 110, 115, 120, 125, 130, 135, 140, 145, 150, 160, 170, 175, 180 minutes of the subject experiencing the trauma/traumatic event. In other examples, HDAC and/or GRK2 inhibitors are administered to the trauma patient within 1 hour, 2, 3, 4, 5, 6, 7, 8, 9, 10, 11, or 12 hours of the subject experiencing the trauma/traumatic event.

### Method of Prophylactically Treating a Subject

Provided herein are methods of prophylactically treating a subject, the method comprising: (a) administering a HDAC inhibitor to the subject; and (b) administering a GRK2 inhibitor to the subject, wherein the subject is at risk of experiencing a clinical trauma.

As used herein, the term “prophylactically treating” can refer to a taking preventative measures to preserve health or prevent the spread of or occurrence of a disease or condition. For example, a subject can be prophylactically treated when the subject is at risk of experiencing a trauma (e.g., having surgery or treatment scheduled, being a soldier in preparation of battle). In some embodiments, the HDAC inhibitor and the GRK2 inhibitor are administered at the same time. In some embodiments, the HDAC inhibitor and the GRK2 inhibitor are administered sequentially. In some embodiments, the HDAC inhibitor is administered before the GRK2 inhibitor is administered. In some embodiments, the HDAC inhibitor is administered after the GRK2 inhibitor is administered. In some embodiments, the HDAC inhibitor is administered before the subject experiences the clinical trauma. In some embodiments, the GRK2 inhibitor is administered before the subject experiences the clinical trauma. In some embodiments, the HDAC inhibitor and the GRK2 inhibitor are both administered before the subject experiences the clinical trauma. In some embodiments, the HDAC inhibitor is administered after the subject experiences the clinical trauma. In some embodiments, the GRK2 inhibitor is administered after the subject experiences the clinical trauma. In some embodiments, the HDAC inhibitor and the GRK2 inhibitor are both administered after the subject experiences the clinical trauma. In some embodiments, the HDAC inhibitor is administered before and after the subject experiences the clinical trauma. In some embodiments, the GRK2 inhibitor is administered before after the subject experiences the clinical trauma. In some embodiments, the HDAC inhibitor and the GRK2 inhibitor are both administered before and after the subject experiences the clinical trauma.

In some embodiments, the subject is at risk of experiencing a clinical, physical, or combat trauma. In some embodiments, the clinical trauma includes surgery, injury, tissue damage, infection, inflammation, pain, medical treatment, secondary disease, or combinations thereof. In some embodiments, the subject is at risk of a physical trauma (e.g., expected wound such as would occur in combat). In some embodiments, the infection is a respiratory infection. In some embodiments, the infection is a nosocomial infection. In some embodiments, the infection is pneumonia. In some embodiments, the infection comprises post-injury pneumonia.

In some embodiments, the HDAC and/or GRK2 inhibitors are administered to the trauma patient before the trauma event occurs. For example, the HDAC and/or GRK2 inhibitors are prophylactically administered to the subject 30 minutes before the subject is expected to experience the trauma/traumatic event. In other examples, the HDAC and/or GRK2 inhibitors are prophylactically administered to the subject 35, 40, 45, 50, 55, 60, 65, 70, 75, 80, 85, 90, 95, 100, 105, 110, 115, 120, 125, 130, 135, 140, 145, 150, 160, 170, 175, 180 minutes before the subject is expected to experience the trauma/traumatic event. In other examples, HDAC and/or GRK2 inhibitors are prophylactically administered to the subject 1 hour, 2, 3, 4, 5, 6, 7, 8, 9, 10, 11, 12, 18, 24, 36, 48, 60, 72, or 96 hours before the subject is expected to experience the trauma/traumatic event. In other embodiments, the HDAC and/or GRK2 inhibitors are prophylactically administered to the subject 1 week, 2, 3, 4, 5, or 6 weeks before the subject is expected to experience the trauma/traumatic event.

### EXAMPLES

The disclosure is further described in the following examples, which do not limit the scope of the disclosure described in the claims.

### Preparation of Human PMN

Human PMN were isolated from freshly withdrawn peripheral blood of healthy volunteers, as described previously. In brief, heparinized whole blood (10 U/ml) was centrifuged at 200×g for 10 min. The buffy coat and RBC were layered onto 1-Step Polymorphs (AN221725, Accurate Chemical & Scientific Corp, Carle Place, NY, USA), followed by centrifugation at 500×g for 30 min. The PMN layer was collected and diluted with an equal volume of 0.45% NaCl to restore osmolarity for 5 min at room temperature. The suspensions were then washed in RPMI 1640 and centrifuged at 200×g for 10 min. RBC in pellet were lysed with ice-cold 0.2% NaCl for 20 sec and the same volume of 1.6% NaCl was applied to end lysis. Cells were centrifuged again at 200×g for 10 min to collect PMN. PMN were resuspended in physiological solutions depending on the next functional study.

### Preparation of Mouse PMN

Mouse bone marrow PMN were isolated from the femurs and tibias of C57/BL6WT or C57/BL6Tlr9−/− as described previously. After euthanizing mice with CO2, the femurs and tibias were harvested. Then, the bone marrow cells were collected and RBC were lysed. PMN were separated from other cells by centrifugation at 2740 rpm for 35 min, RT on gradients (Histopaque-1077 and Histopaque-1119, MilliporeSigma, Burlington, MA, USA). Collected PMN were washed twice with RPMI supplemented with 10% FBS and 1% penicillin/streptomycin.

Preparation of mtDNA

Human or mouse mtDNA was prepared directly from the grossly normal pathologic margins of operative liver resection specimens. Tissues were processed using an mtDNA Extractor CT Kit (Fujifilm/Wako, Richmond, VA, USA) following the manufacturer's methods. mtDNA concentration was confirmed by NanoDrop 2000 and qPCR analysis against human CYTB or mouse Cytb.

CTX assay PMN CTX was studied in 3.0 m-pore-transwells. 1×10⁵ PMN in 75 μL of RPMI with 2% heat-inactivated FBS were applied to the upper chamber and 150 μL of the same media containing indicated chemoattractants were applied to the lower chamber. mtDNA application was done at RT, rotating for 15 min. When stated, methyl 5-[2-(5-nitro-2-furyl)vinyl]-2-furoate (GRKi), paroxetine (PAR) or valproic acid (VPA) were treated by the same method for 30 min in prior to mtDNA treatment. Cells were incubated (37° C., 5% CO2) for 60 min. PMN were then collected from the lower chambers, centrifuged (500×g, 5 min, RT) and re-suspended in 200 μL of cell lysis solution (×1/20) with CyQUANT GR dye (×1/400) in water for 15 min at RT (C7026, Thermo Fisher Scientific). PMN numbers were evaluated in 96-well plates using 480 nm excitation and 520 nm emission with known numbers of PMN for standard curve.

### Flow Cytometry for Surface Receptor Expression Analysis

PMN were treated with 20 μM PAR, 1 mM VPA or carrier for 30 min at RT rotating before being treated with or without 40 μg/mL mtDNA for 5, 15 or 60 min at RT rotating. Finally, PMN were incubated with fluorescein (FITC)-conjugated anti-human FPR1 antibody (FAB3744F, LifeSpan BioSciences Inc., Seattle, WA), phycoerythrin (PE)-conjugated anti-human CXCR2 antibody (FAB331P, LifeSpan BioSciences Inc.), or PE-conjugated anti-human BLT1 antibody (FAB099P, LifeSpan BioSciences Inc.) in the dark for 30 min at room temperature. FITC-conjugated isotype antibody (400210, BioLegend, San Diego, CA) or PE-conjugated isotype antibody (400212, BioLegend) was used as a negative control. Allophycocyanin (APC)—conjugated anti-human CD16 antibody (360705, BioLegend) was used to confirm PMN identification.

### Western Blots

Briefly, PMN samples were dissolved in Pierce RIPA lysis buffer (25 mM Tris HCl pH 7.6, 150 mM NaCl, 1% NP-40, 1% sodium deoxycholate, 0.1% SDS) (Thermo Fisher Scientific, Waltham, MA, USA) containing Halt Protease & Phosphatase Inhibitor Cocktail (Thermo Fisher Scientific) for 20 min on ice, followed by centrifugation at 14000 for 20 min at 4° C. Then, the supernatant was collected, concentration was measured using a Pierce BCA Protein Assay Kit (Thermo Fisher Scientific). After mixing and boiling (95° C., 10 min) protein lysate with appropriate amount of 6×Laemmli SDS-sample buffer (J61337, Alfa Aesar, Tewksbury, MA, USA) according to the protein concentration, 40 μg of protein lysates were separated by SDS/PAGE and transferred to nitrocellulose membranes contained in an iBlot Transfer Stack kit (IB301002, Thermo Fisher Scientific). The membranes were blocked with 5% skim milk in PBST for 1 hr at RT, and then incubated with the primary antibody against GRK (MAB43391, R&D Systems, Inc.), phospho-GRKSer670 (PA5-77851, Thermo Fisher Scientific) or 3-actin (sc-47778, Santa Cruz Biotechnology, Santa Cruz, CA, USA) overnight at 4° C. After being incubated with their appropriate secondary antibodies conjugated with horseradish peroxidase (HRP) the following day, the immunoblot signals were detected by application of the Amersham ECL Prime Western Blotting Detection Reagent kit (Cytiva, Marlborough, MA, USA) in a ChemiDoc Imaging System (Bio-Rad, Hercules, CA, USA). Quantification of blots were performed using Image J v.1.53i (National Institute of Health, Bethesda, MD, USA).

### Calcium Measurements by Spectrofluorometry

Freshly isolated human PMN were incubated in 2 μM fura 2-AM (F1201, Thermo Fisher Scientific) at 37° C. for 45 min in dark. Specimens were divided into aliquots of 5×10⁵ cells and kept on ice in dark, and were incubated at 37° C. for 5 min just before each experiment. PMN were then pelleted by centrifugation (5000 rpm, 30 sec) and resuspended in a cuvette containing 3 mL HEPES that consisted of the following: NaCl 140 mM, KCl 5 mM, MgC12 1 mM, glucose 100 mM, Hepes 500 mM (pH 7.4) with 0.1% BSA. Experiments were generally begun in nominally calcium-free medium containing 0.3 mM EGTA. Intracellular calcium was monitored by measuring fluorescence at 505 nm, using 340/380-nm dual wavelength excitation in a Fluoromax-3 spectrofluorometer (Horiba Jobin Yvon, Piscataway, NJ). Cuvette temperatures were kept at 37° C. with constant stirring. Calibration was performed at the end of each experiment by addition of 100 μM digitonin (Molecular Probes) and then 15 mM EGTA to achieve RMAX and RMIN. The autofluorescence of a sample cell suspension treated with 100 M digitonin and 2 μM MnC12 was subtracted from total fluorescence. The [Ca²⁺ ]i was then calculated from the 340/380 nm fluorescence ratio (Kd=220 nM) according to the methods of Grynkiewicz et al.

### Respiratory Burst Assay by Reactive Oxygen Species (ROS) Measurement

ROS production was measured by luminol-dependent chemiluminescence in a 96-well plate luminometer (LB 960, Berthold). Briefly, PMN were pretreated with 40 μg/mL mtDNA or the same amount of TE (vehicle) for 15 min at RT with rotation. For ROS measurement, PMN (4×10⁶ cells/mL) were mixed 1:1 with 2×detection reagent consisted of 0.2 mM luminol (Sigma, 123072) and 150 nM HRP in DPBS+(14040117, Gibco) and then pre-warmed at 37° C. for 5 min. After pre-warming, 180 μL of PMN/detection reagent mix was transferred manually in quadruplicate to a pre-warmed 96-well plate and chemiluminescent detection was started. At the indicated time points, 20 μL of DPBS+(resting cells), PMA (5 μM, positive control), or chemoattractants (fMLF 100 nM, ND6 100 nM, LTB4 10 nM) were injected automatically by the luminometer. Output is displayed in relative light units per second (RLU/sec) and quantification was calculated by the area under curve (AUC) of RLU/sec.

### Bacterial Phagocytosis Assay by Colony-Forming Unit (CFU) Count

Before the CFU count of bacteria, Staphylococcus aureus (Sa) were opsonized with autologous serum from the PMN donor. OD₆₀₀=0.1 were determined as 108 CFU/mL according to previous measurements. Freshly isolated PMN were treated with PAR or VPA for 30 min at RT rotating and mtDNA was treated for 15 min at RT rotating following PAR or VPA. Then, Sa were incubated with PMN at 1:1 ratio in 1 mL 0.9% NaCl supplemented with human serum for 30 min rotating. The PMN were pelleted by 500×g centrifugation for 5 min and lysed by 0.05% saponin in 0.9% NaCl to harvest phagocytosed bacteria. The supernatant was collected separately, centrifuged at 5000×g for 10 min to pellet bacteria. The bacteria were applied on agar plates and after 18 hrs the bacterial colonies were counted.

### Bacterial Phagocytosis Assay by Flow Cytometry

Sα were labeled with SYTO9 (ThermoFisher Scientific) for 10 min at RT in dark. The remaining stain was removed after centrifugation at 5000×g for 10 min. PAR or VPA was treated to PMN for 30 min and mtDNA was treated for following 15 min rotating at RT. Then, SYTO9-labeled Sa (4×10⁶ CFU/mL) were incubated with PMN (4×10⁶ cells/mL) at 1:1 ratio in 1 mL 0.9% NaCl supplemented with human serum for 30 min rotating. The PMN were pelleted by centrifugation at 500×g for 5 min and then resuspended in 200 μL FACS buffer and stained with allophycocyanin (APC)—conjugated anti-human CD16 antibody (360705, BioLegend) to confirm PMN identification.

### Actin Polymerization Assay by Rhodamine Phalloidin Staining

Actin polymerization was studied using an F-Actin Visualization Biochem Kit (Cytoskeleton, Inc., Denver, CO, USA) according to the manufacturer's instructions. Briefly, PMN were applied to glass slides (Thermo Fisher Scientific) pre-coated with 0.1% poly-L-lysine solution (P8920, MilliporeSigma) in H2O. Then 200 μL of Fixative solution was added to the slides and incubated for 10 min at RT. After washing with 200 μL of Wash buffer for 30 sec at RT, 200 μL of Permeabilization Buffer was added to the slides and incubated for 5 min at RT. Then, the cells were stained with rhodamine phalloidin for 30 min at RT in dark. The slides were then washed with Wash buffer and Hoechst 33342 (1:1,000 in PBS) was applied to stain nucleus. Followed by 3 times of washing, 20 μL of Mounting medium was added to the center of each slide and a coverslip was put on. Clear nail polish was used to seal coverslips. The actin filaments were observed using AxioImager Epifluorescence Microscope (Zeiss, Jena, Germany) at excitation 535 nm and emission 585 nm.

### Animal Studies

Following 48 hr acclimatization, 8-9 week-old male CD-1 mice (body weight 30-32 g) underwent laparotomy followed by crushing of the left lobe of liver 8 times using a sterile forceps. After 4 h, the trachea was exposed under anesthesia and Sa (1.8×108 CFU in 50 μL PBS) were applied intratracheally with a 30 g needle. PAR (20 mg/kg), VPA (80 mg/kg) or a combination of the two were injected i.p. 30 min before or after the laparotomy as indicated. Mice were sacrificed 18 h later and bronchoalveolar lavage (BAL) fluid (BALF) was collected by tracheal irrigation with 1 mL of PBS. The collected BALF was centrifuged; the pellet was diluted to count the number of immune cells; and the supernatant was then separated and applied to agar plates to count the remaining bacteria. Experiments were repeated as noted and data were combined.

### Statistical Analysis

Quantitative data were expressed as mean±standard error of mean (SEM) for 3 or more independent experiments as noted. Statistical analysis was performed using Prism 8 (GraphPad, San Diego, CA). Data were analyzed by analysis of variance (ANOVA) followed by Tukey's post hoc test. Probability (p) values less than 0.05 were considered statistically significant.

Example 1—mtDNA Suppresses PMN Chemotaxis Via Endosomal TLR9

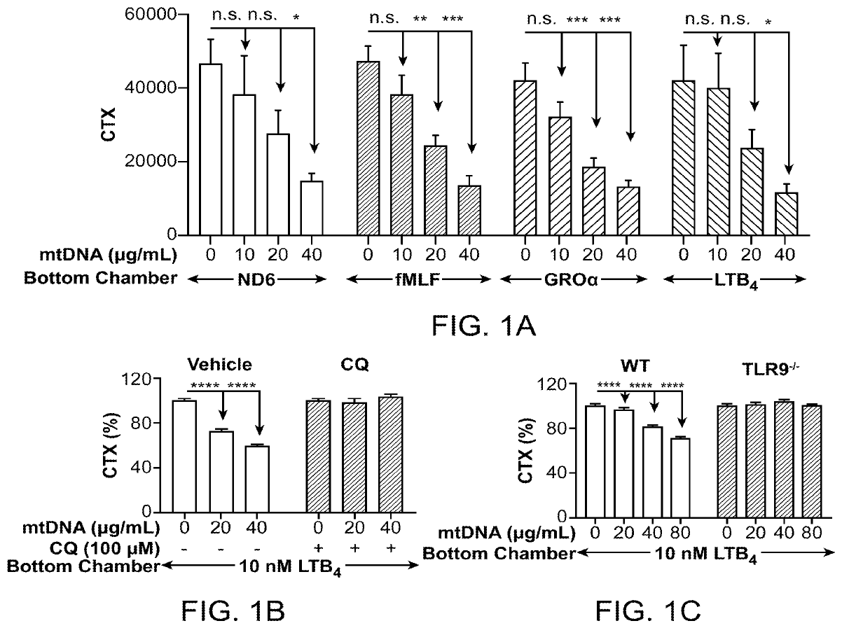

It was confirmed that mtDNA suppresses chemotaxis to multiple GPCR stimuli including mitochondrial and bacterial formyl peptides (ND6, fMLF), chemokines (GRO-α/CXCL1) and lipid agonists (LTB4) in dose-dependent fashions (FIG. 1A); that the suppressive effect of mtDNA is blocked by CQ, showing dependence on endosomal acidification (FIG. 1B) and that the suppressive effects of mtDNA were absent in PMN from TLR9−/− mice (FIG. 1C).

Example 2—mtDNA does not Change GCPR Expression or Receptor Bias

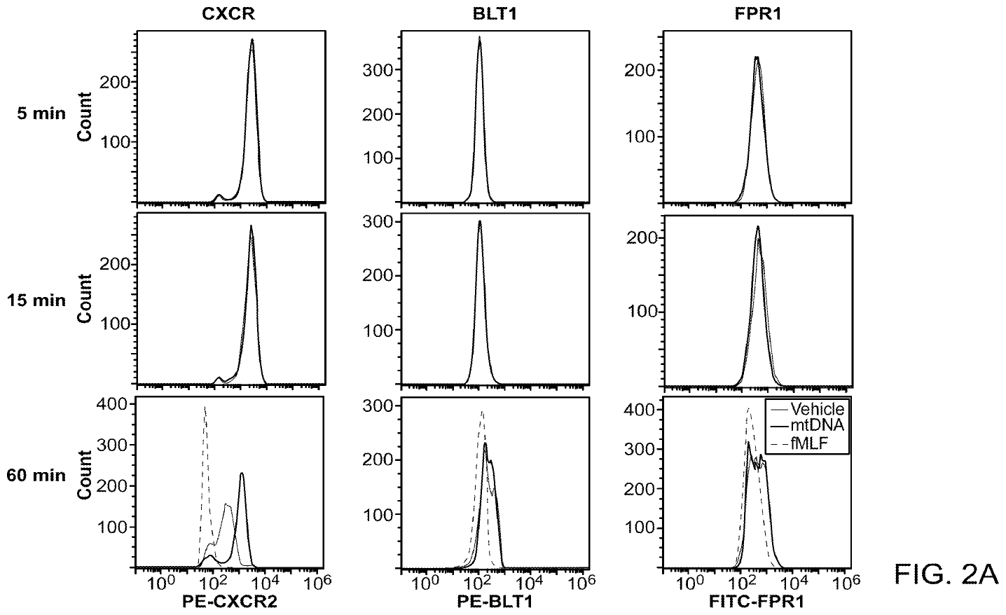

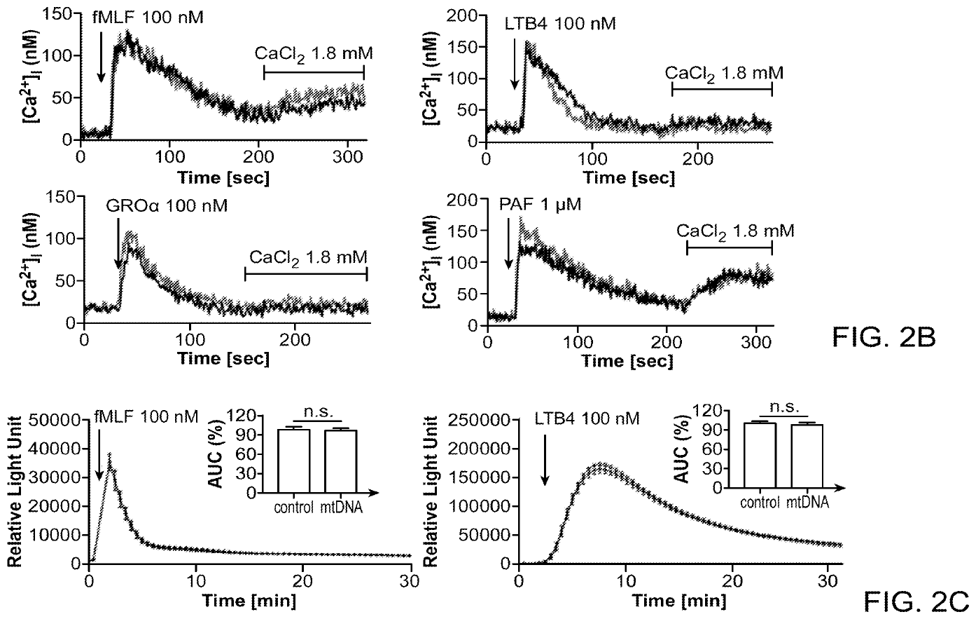

Next, GPCR expression and functional bias was evaluated. In FIG. 2, it is shown that mtDNA does not change either GCPR expression or functional bias: (FIG. 2A) mtDNA fails to suppress human PMN surface expression of FPR1, BLT1 and CXCR2 at 5 and 15 minutes. At 60 minutes there was an increase in CXCR2 expression. All GPCRs studied were regulated by fMLF (third row). (FIG. 2B) Cytosolic calcium ([Ca₂₊]i) responses to fMLF, LTB₄, GROα, and PAF in Ca²⁺-free and then Ca²⁺-replete media were identical without (black trace) and with (red trace) prior exposure to mtDNA. (FIG. 2C) Receptor dependent respiratory burst (RB) responses to fMLF and LTB4 were unaffected by mtDNA. N=3-4 for all experiments.

Example 3—mtDNA- and FP-Induced Suppression of CTX Depends on GRK2

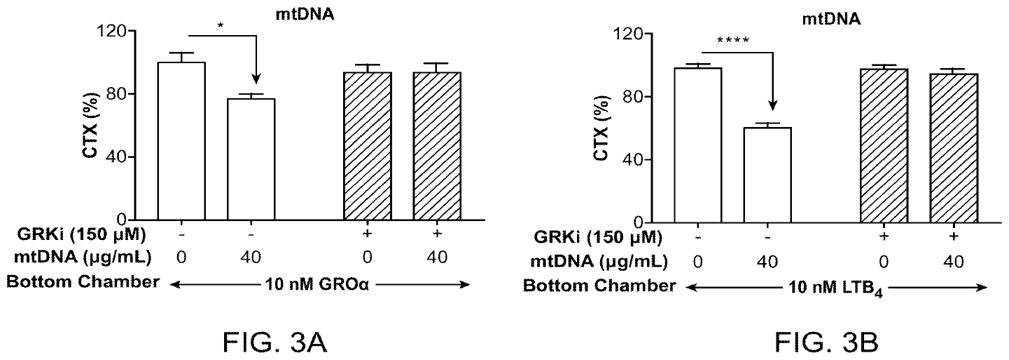

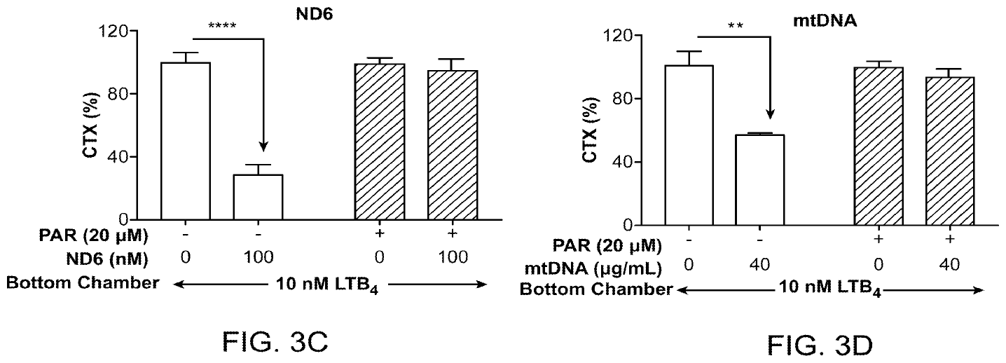

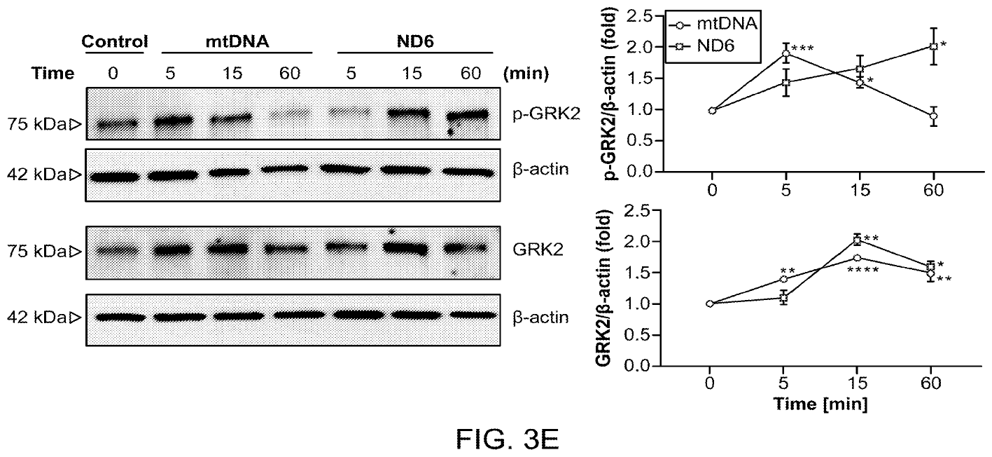

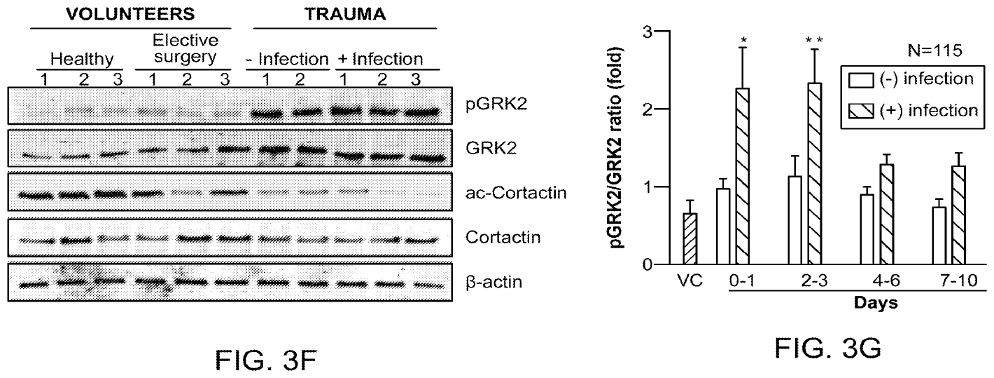

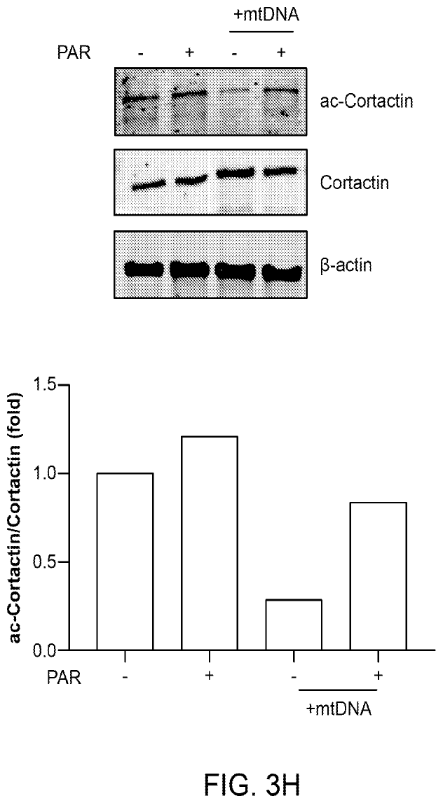

GPCR regulation is commonly associated with activation of G-protein receptor kinases (GRKs) by phosphorylation and GRK2 is a dominant GRK in PMN. So despite the failure of GPCRs to show expression or bias changes, PMN was examined for evidence of mtDNA dependent GRK2 activation. GRK2 activation by FPs (ND6) was studied as a positive control. FIG. 3 shows GRK2 activation by mtDNA and FPs in PMN: (FIG. 3A) Western blots show mtDNA and ND6 each cause both increased GRK2 phosphorylation and increased GRK2 protein expression. The time courses of phosphorylation and expression changes (line graphs) however, were distinctly different after stimulation by ND6 (via FPR1) versus by mtDNA (via TLR9). mtDNA-induced suppression of CTX to both GRO-α (FIG. 3B) and LTB4 (FIG. 3C) were rescued by the GRK2 inhibitor methyl 5-[2-(5-nitro-2-furyl)vinyl]-2-furoate (GRKi). Suppression of both PMN CTX to LTB4 after ND6 exposure (FIG. 3D) and CTX to LTB4 after mtDNA exposure (FIG. 3E) were also rescued by the GRK2 inhibitor Paroxetine (PAR). All changes were significant (ANOVA/Tukey's test. N=6 for all experiments.). Healthy volunteer PMNs show very low baseline Ser685 phosphorylation (activation) of GRK2, PMN from trauma patients show many-fold increased GRK2 activation (FIG. 3F). This activation peaks 1-3 days after injury (FIG. 3G). More remarkably, those trauma patients developing pneumonia showed far more GRK2 activation than trauma patients who did not develop infection. (FIG. 3F). Further, HDAC6 activation acetylates cortactin (CTTN). CTTN is responsible for actin cytoskeletal branching and elongation and this activity is inhibited by acetylation. Paroxetine blocks mtDNA suppression of cortactin acetylation (see FIG. 311), and further valproate (VAL) blocks activation of HDAC6, explaining why VAL+PAR rescues F-actin polymerization.

Example 4—mtDNA Decreases PMN Bacterial Phagocytosis

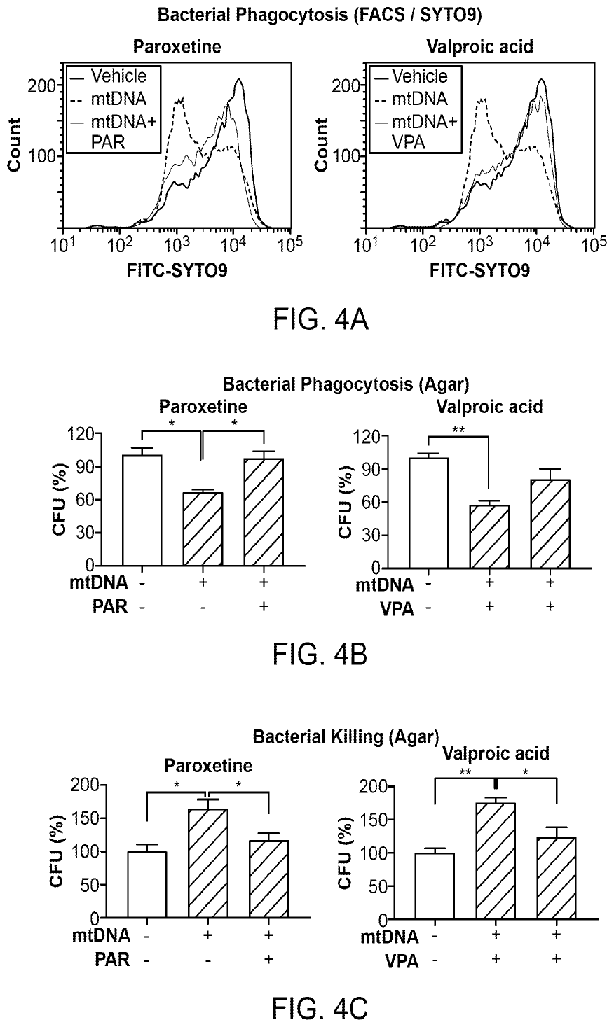

Having shown that mtDNA activates GRK2 and that GRK2 inhibition restores PMN chemotaxis, it was next evaluated whether GRK inhibition could restore any other mtDNA-induced suppression of human PMN microbial functions. FIGs 4A and 4B show mtDNA decreases PMN phagocytosis of Staphylococcus aureus (Sa). Flow cytometry shows that phagocytosis of SYTO9 labeled Sα by human PMN (FIG. 4A, PMN labelled with CD16+) is suppressed by mtDNA. There is a suggestion that two PMN populations are created by that mtDNA exposure. PAR restored phagocytosis as seen on flow cytometry (FIG. 4A) and on an agar plate assays (FIG. 4B). PAR also restored Sa killing (4C) in co-culture with PMN. Because mtDNA did not regulate receptors (FIG. 2A) and it has been suggested GRK2 can act through histamine deacetylase 6 (HDAC6), the HDAC inhibitor Valproic Acid (VPA) was also assessed and found that it could also reverse mtDNA-mediated suppressive effects on bacterial phagocytosis and killing (FIG. 4A-4C).

Example 5—Inhibition of F-actin polymerization by mtDNA is rescued by PAR and VPA

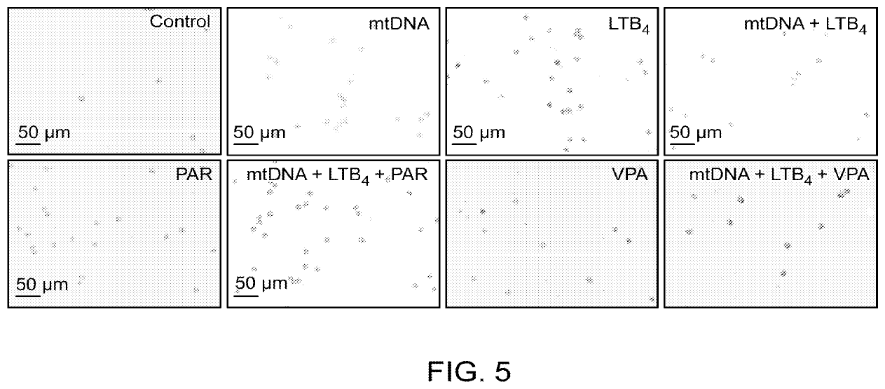

Chemotaxis and phagocytosis both depend on function of the actin cytoskeleton which is impaired in sepsis by GRK2. Thus, because a) mtDNA activates GRK2 (FIG. 3), b) GRK2 acts on HDAC6 and c) HDACs modulate microtubular organization through acetylation of cortactin (CTTN), human PMN was evaluated for any potential effects of mtDNA on cytoskeletal function and to evaluate whether PAR and or VPA might be able to modify any such effects seen. In FIG. 5 it is shown that LTB4 causes brisk G-actin polymerization to F-actin (upper panel 3) as compared to control (upper panel 1). It is then noted that mtDNA strikingly inhibits polymerization of F-actin (upper panel 4). Cytoskeletal responses were rescued by either PAR or VPA (lower panels 2 and 4). No significant actin reorganization was noted in the absence of LTB4 (Rhodamine-phalloidin stain, 200×, scale bar=50 m).

Example 6—In-Vivo Effects of Single Vs Dual GRK Pathway Inhibition on Infection after Trauma

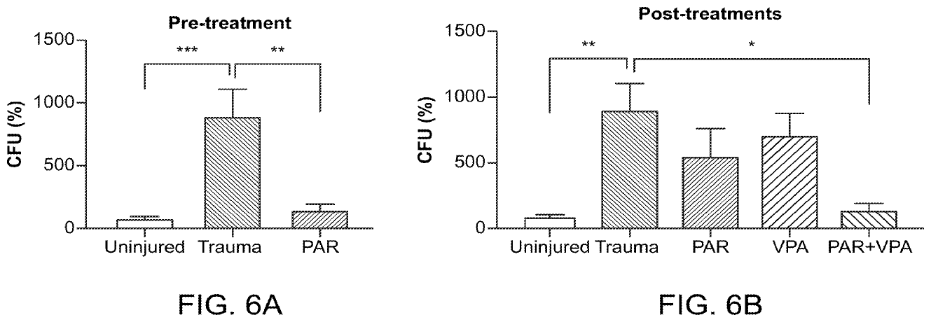

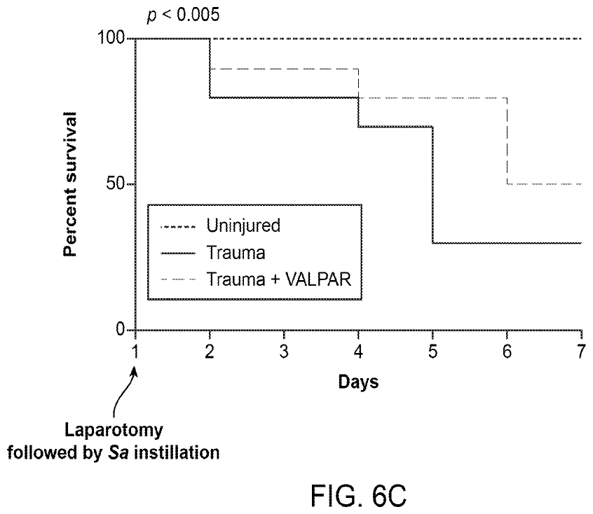

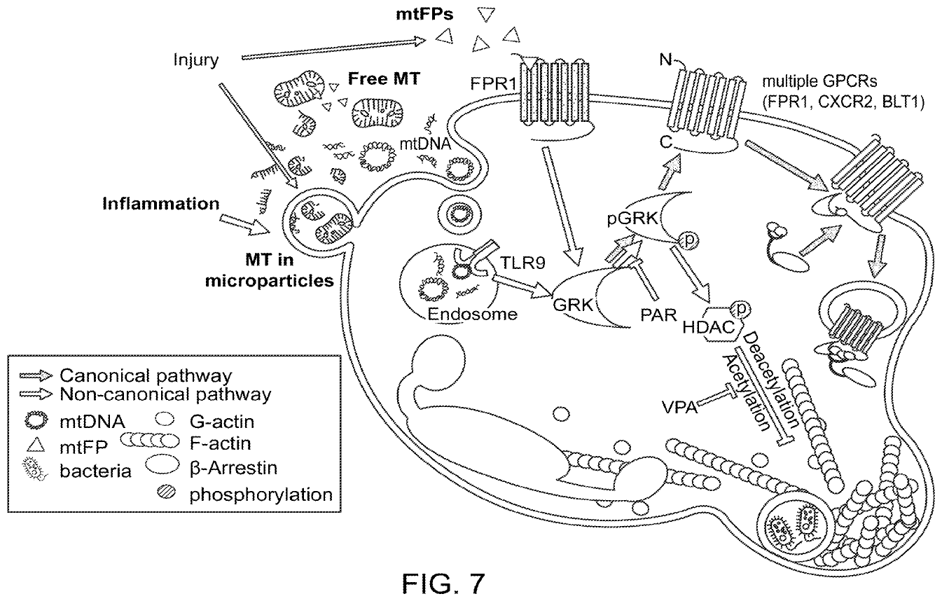

The examples above showed 1) that mitochondrial DAMPs suppress PMN function via two distinct GRK-related pathways; 2) that GRK2 activation suppresses microtubular reorganization; 3) that mtDNA-initiated GRK2 activation disrupts multiple PMN antimicrobial functions; and 4) that GRK and HDAC inhibitors can reverse some or all of these defects in-vitro. It was next studied whether GRK inhibition might reverse injury-induced suppression of antimicrobial function in-vivo. For this a model of systemic trauma-dependent pulmonary infection in mice was used. In FIG. 6A it is shown that uninjured CD-1 mice (left) normally clear bacteria (1.8×10⁸Sα injected intra-tracheal [i.t.]) overnight. Animals undergoing Trauma (laparotomy+liver crush) 4 hours before Sa injection clear the inoculum very poorly (center). Pre-treatment with PAR (20 mg/Kg, 30 min prior to injury) markedly improved bacterial clearance (right).

The timing of trauma is not predictable in clinical practice though. So pre-treatment studies are often not useful guides to clinical use. The effects of GRK-pathway inhibitors given after injury was then studied, i.e. as a post-treatment. When given 30 minutes post-injury (FIG. 6B), it was found that single-drug therapies given at clinical doses (PAR 20 mg/kg or VPA 80 mg/kg) did not confer significant protection (Bars 3, 4). In contrast, PAR and VPA given at the same doses in combination after trauma+infection rescued lung bacterial clearance back to the level of uninjured controls (Bar 5). N=13 for uninjured control and trauma groups. N=6-10 for treatment groups. CFU denotes colony forming units of Sa retrieved by broncho-alveolar lavage (BAL) and presented as percent of the uninjured control values. The VAL+PAR combination improved the survival of injured mice inoculated with Staph aureus intratracheally (FIG. 6C).

### Example 7—G-Protein Kinase Inhibition and Anti-Infective Function

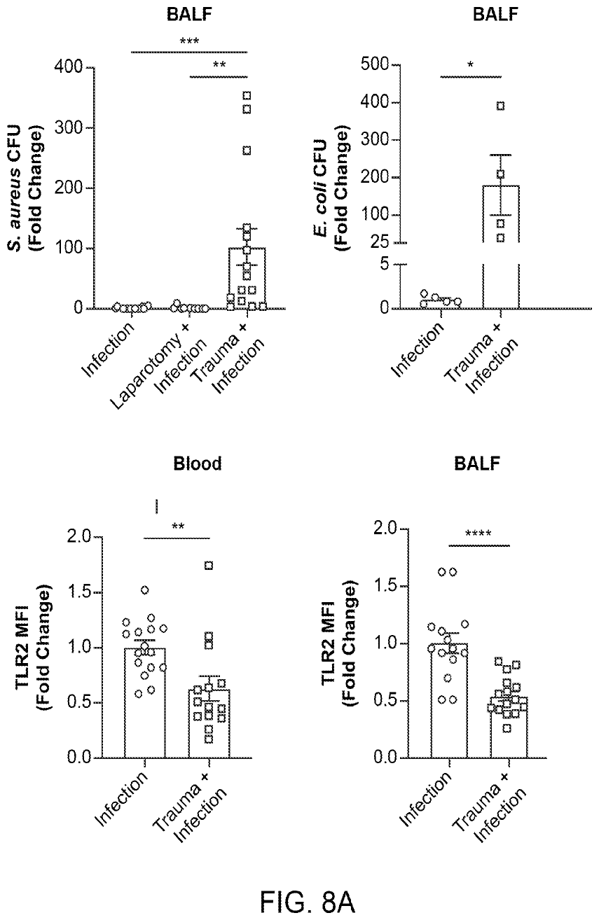

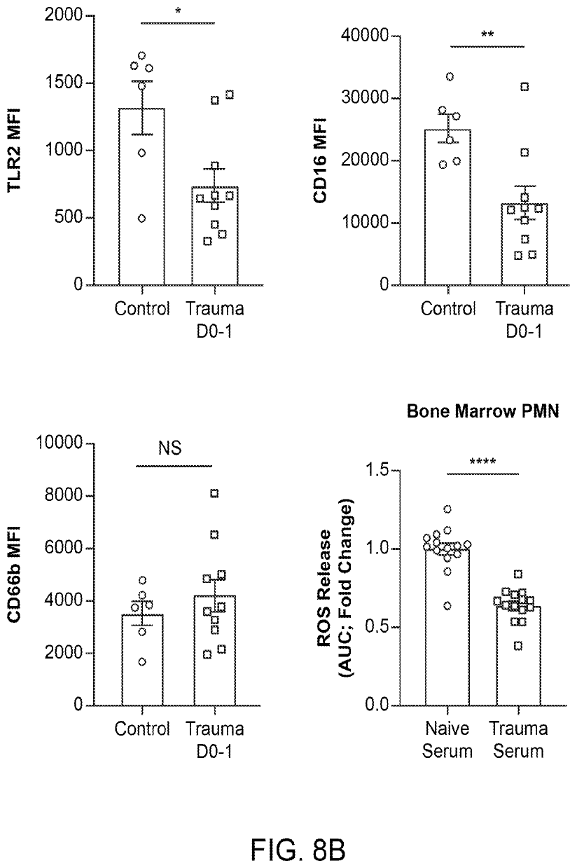

Clinical injury can predispose a subject to infection. It has been validated that nearly identical innate immune responses to trauma are seen in rodents, pigs, and humans, where injury increases susceptibility to lung inoculation by ubiquitous bacteria. FIG. 8 shows that PMN from traumatized mice and humans are similar in that they have decreased Toll-like-Receptor-2 (TLR2) expression. TLR2 is critical for S. aureus recognition. Moreover, mouse PMN incubated with trauma plasma showed decreased respiratory burst in response to bacteria compared to PMN exposed to naïve mouse plasma. Although leukocyte dysfunction involves multiple signaling pathways, we have demonstrated that PMN exposed to trauma plasma are ineffective at recognizing and killing bacteria across species.

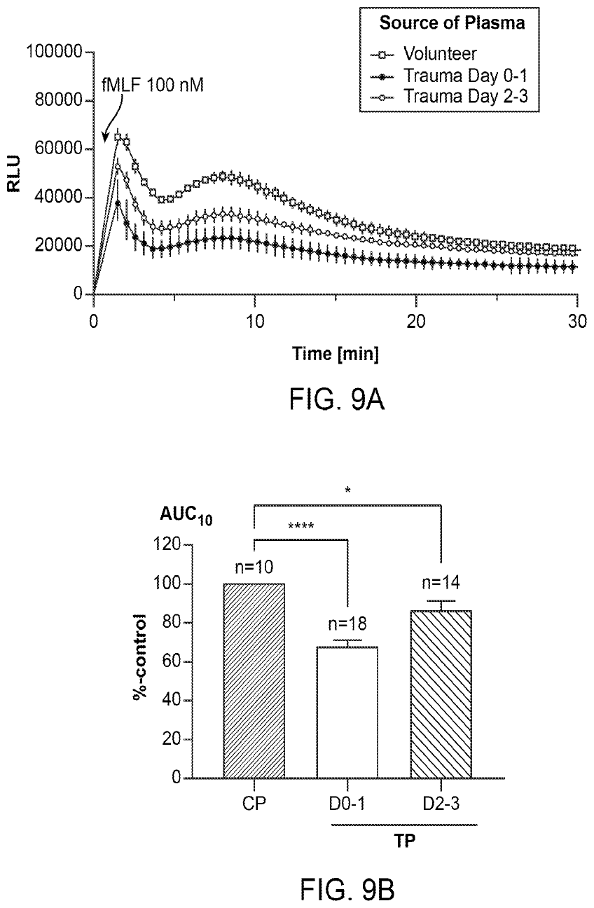

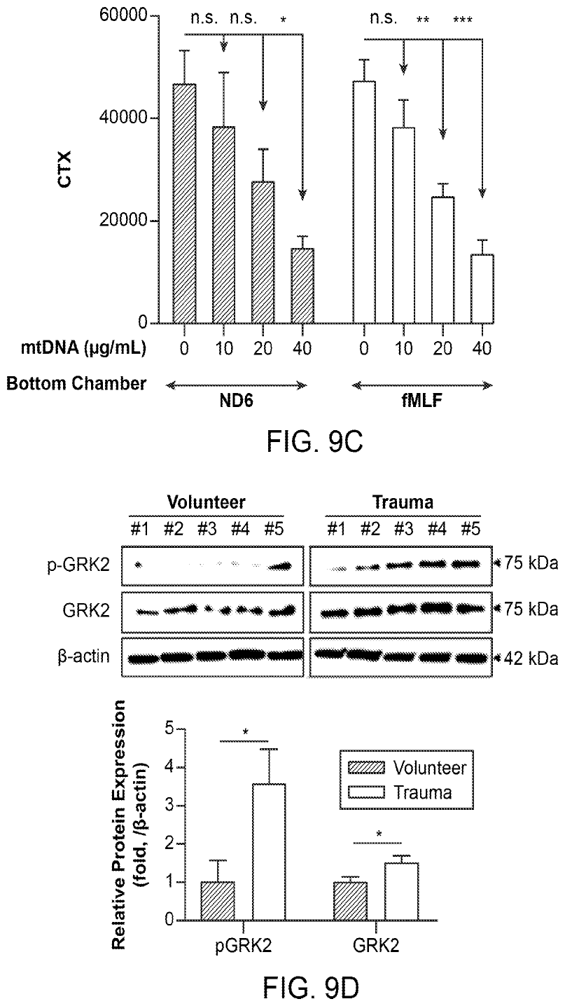

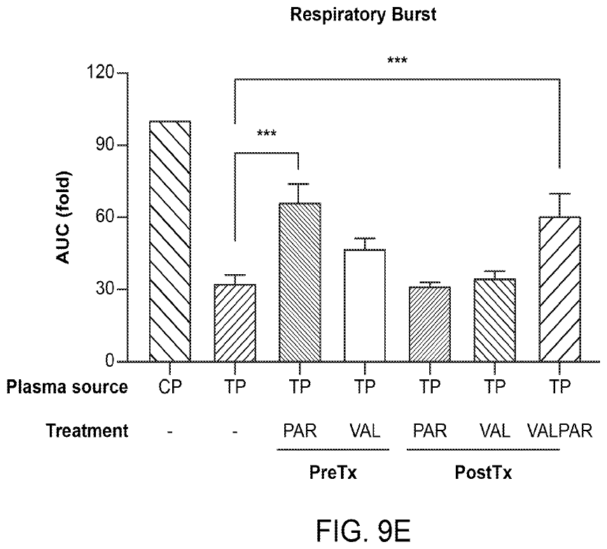

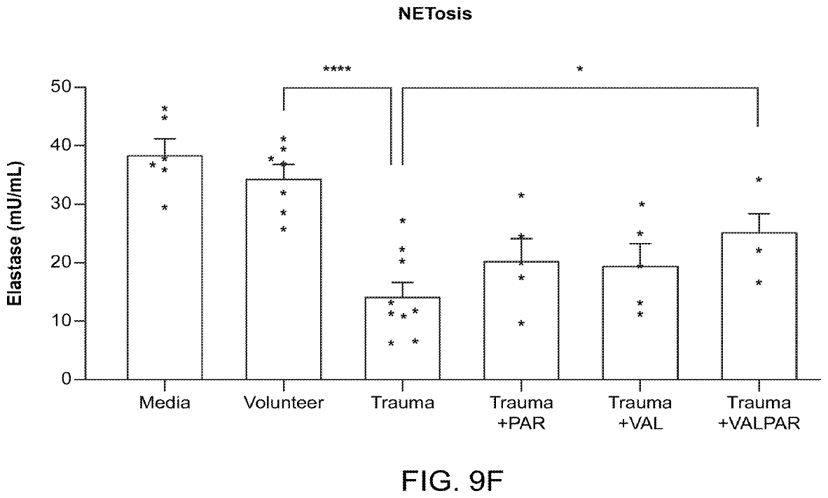

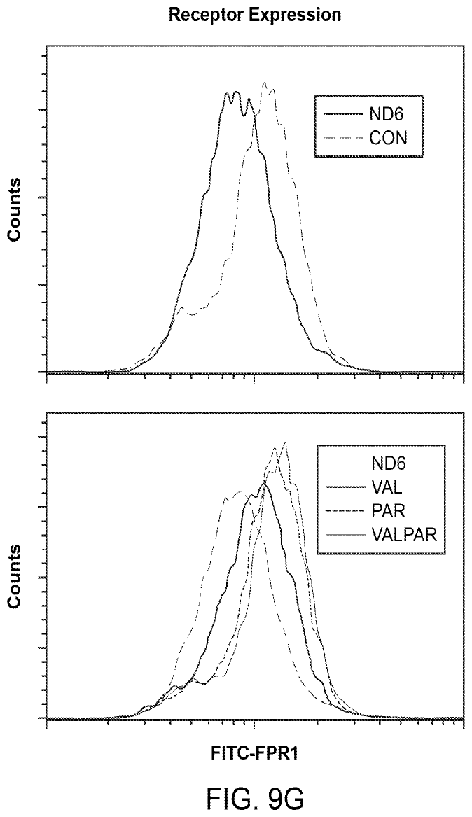

Extensive mechanistic studies were performed supporting this transformative therapeutic approach to respiratory health. Aspects studied include chemotaxis, respiratory burst (FIGS. 9A-9C and 9E) as well as phagocytosis, PMN extracellular trap formation (NETosis) (FIG. 9F), bacterial killing, and Formyl Peptide Receptor-1 (FPR1) expression (FIG. 9G). In studying the mechanisms underlying such PMN suppression, we discovered that inhibition of PMN effector functions in response to DAMP exposure after trauma is due in significant measure to the pathologic activation of G-Protein Coupled Receptor Kinases (GRKs) (FIG. 9D).

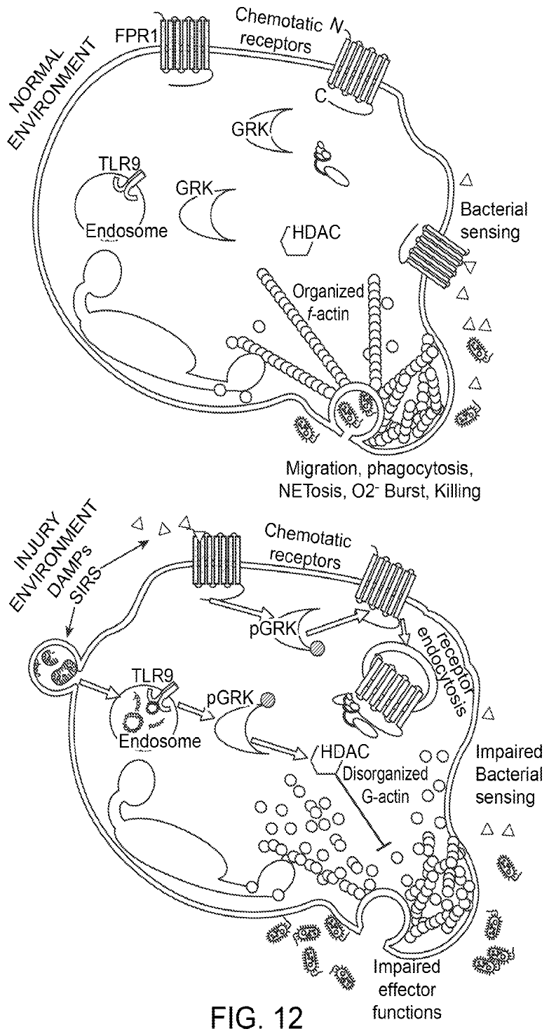

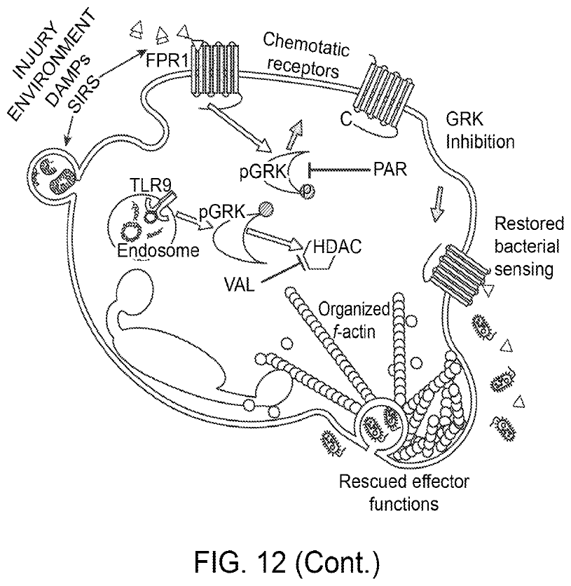

GRK activation leads to the well-described ‘canonical’ internalization of G-Protein Coupled Receptors (GPCRs) that are critical sensors of infective environments. It was also discovered that mtDAMP activation of PMN toll-like receptors (TLRs) and specifically TLR9, causes GRK2 to activate Histone Deacetylases (HDACs) as downstream targets, and shown that such HDAC activation results in the disruption of actin polymerization (FIG. 5). Cytoskeletal dysfunction is a critical determinant of suppressed PMN migration and phagocytosis. These findings therefore led to a working hypothesis that the post-traumatic trauma plasma SIRS environment suppresses PMN activity via a dual GRK2-mediated pathway inhibition (FIG. 12).

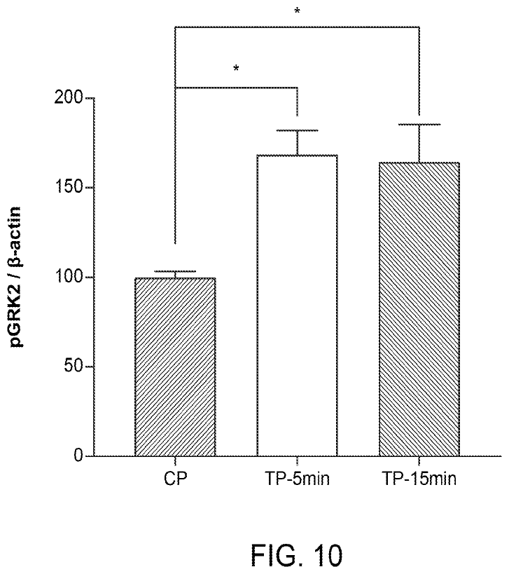

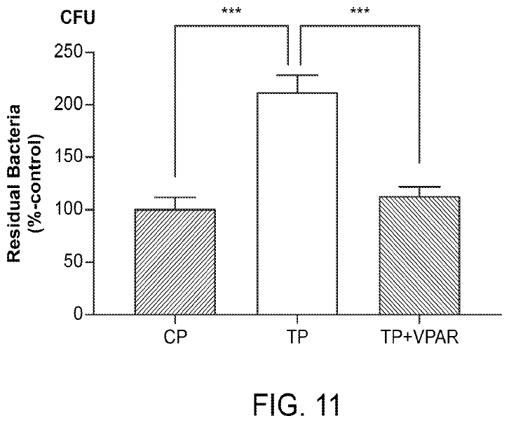

To demonstrate that this pattern of dysfunctional cell signaling was indeed the result of the inflammatory ‘systemic inflammation responses to the injury (SIRS)’ environment of trauma, it was next shown that normal (volunteer) PMN clearly exhibit GRK2 activation after exposure to trauma patient plasmas (FIG. 10). To further confirm the relevance of these signaling events, it was shown that PMN exposure either to the same clinical trauma patients' plasma samples or to purified DAMPs suppresses multiple key anti-microbial effector functions (FIG. 4A). Moreover, all of those suppressive events could be reversed by a variety of pharmacologic GRK2 inhibitors or by inhibition of HDAC signaling. But the reversal is only partial unless both GRK2 and HDAC are inhibited (FIG. 11). The specific GRK2 and HDAC combination of PAR/VAL was studied in-vivo in a relevant mouse model of injury (liver crush) plus lung bacterial inoculation. Here it was found that pre-injury use of VAL or PAR returns post-injury pulmonary bacterial clearance towards pre-injury levels. Since all therapies used in trauma are used after the injury, it was found that when used together post-injury treatment rescues lung bacterial clearance to the levels seen in uninjured mice (FIG. 6B).

Example 8—Two-Hit Model of Lung Infection after Trauma in Pigs

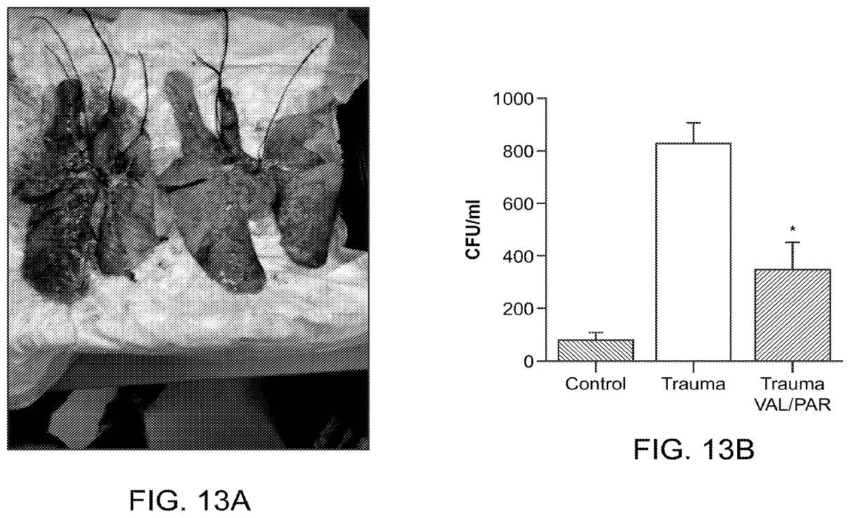

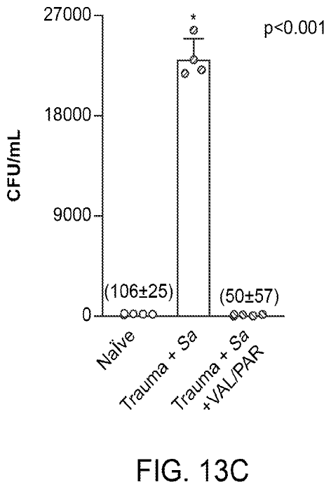

A pig model was developed involving instilling a homogenate of 10% (by weight) of normal donor pig liver via mini-laparotomy into the abdomen of male or female Yorkshire pigs (25 kg). The liver homogenate was derived from a syngeneic donor pig under sterile conditions. Forty-eight hours later, the pigs were inoculated with 108 cfu Gram+S. aureus in 15 ml of saline into both the right cranial lobe and right lower lobe via bronchoscopy. The animals were allowed to recover and lungs harvested at 24h later. A bronchoalveolar lavage (BAL) is performed ex vivo. The lung tissue (each lobe) is then divided into distal, medial, and proximal sections and homogenized. After centrifugation of the homogenate and BAL fluid, aliquots of the supernatants were analyzed for bacterial counts by agar plate assay. Cell counts were determined in the BAL and whole blood and profiled using specific pig antibodies for neutrophils (mature vs immature) and macrophages (M1 vs M2) using CyTOF and/or FACs analyses. FIG. 13B shows preliminary data that VAL+PAR can markedly reduce injury-induced susceptibility to S. aureus lung infection. FIG. 13C shows updated data that VAL+PAR can rescue trauma-induced deficits in bacterial clearance. The data in FIG. 13 used clinically relevant doses and routes of administration.

### Example 9—Additional Studies of VAL, PAR, and VAL+PAR in Pigs

Additional experiments are planned using the Gram+S. aureus. Further, given that Gram-strains can also lead to pneumonia and acute lung injury, and that Pseudomonas aeruginosa is a commonly encountered multi-drug resistant organism in trauma patients, the ability of VAL+PAR to rescue injury-induced susceptibility to lung inoculation by P. aeruginosa will also be tested. Ninety minutes after the liver injury plus hemorrhage, bacteria (S. Aureus (SA) or P. aeruginosa (PA) are instilled into the right lung by bronchoscopy (5×10⁷⁻⁹ cfu/each into the right upper [cranial] and right lower lobes). Pigs are resuscitated with LR at 2×shed blood volume 6 hours later. Paroxetine (PAR)+Valproate (VAL) (or vehicles) are given p.o. 1-4h post injury and daily thereafter through an indwelling jugular line, using doses of 0.3-1 mg/kg PAR and 15-100 mg/Kg VAL. Pigs are sacrificed and lungs excised at 24 h, 48h, 72h and 1 wk after inoculation. Lung and blood samples are assayed for bacterial counts, inflammation (cytokines) and lung injury (pathology).

VAL will be administered at doses of 10, 30 and 100 mg/kg delivered enterally by PEG placed at the time of laparotomy. Similarly, PAR will be dosed daily at 0.1, 0.3 and 1 mg/kg by PEG. Controls will consist of pigs subjected to trauma+infection+saline (n=24 males and females for individual dosing and 48 for combination dosing). The first dose of each will be administered just after closing of abdomen after liver injury induction. Combination therapy will comprise administering each, one after the other and will be tested as described in Table 1, below. All pigs will be monitored for any overt adverse events.

TABLE 1

| Individual Dosing | | | | |
|---|---|---|---|---|
| Valproate | Paroxetine | Combination | | |
| (mg/kg) | (mg/kg) | Val + Par (mg/kg) | | |
| 10 | 0.1 | 10 + 0.1 | 30 + 0.1 | 100 + 0.1 |
| 30 | 0.3 | 10 + 0.3 | 30 + 0.3 | 100 + 0.3 |
| 100 | 1.0 | 10 + 1.0 | 30 + 1.0 | 100 + 1.0 |

| | |
|---|---|
| Total Pigs 49 (male/female) | Total Pigs 96 (male/female) |

Samples will be collected for cytokines and measurements of stress response genes in the tissue homogenates for nrf2, pGRK2/GRK2, cortactin/acetyl-cortactin, HDAC6, and HO-1 by Western blot and PCR. Plasma samples will be assayed for mtDNA, heme, and formyl peptides. Additional aliquots of plasma will be assayed for GM-CSF, IFNgamma, IL-1alpha, IL-1ra, IL-1beta, IL-2, IL-4, IL-6, IL-8, IL-10, IL-12, IL-18, and TNF. Additional tissue samples will be analyzed for bacterial counts.

Example 10—Efficacy of VAL, PAR, and VAL+PAR in ICU patients

Intubated trauma patients who have received 1 unit of packed red blood cells (RBC) in transfusion will be recruited at the time of admission. Venous, arterial, and enteral access as well as EKG monitoring will be required. Eighty patients will be randomized to 4 groups receiving VAL, PAR, VAL+PAR, or placebo on hospital days 1-3. Bioavailability of both VAL and PAR are excellent both by parenteral and enteral routes, with no significant differences found in multiple studies with no drug-drug interactions seen in clinical settings. Thus, VAL and PAR will administered via the enteral route. Between 0.1-1.0 mg/Kg paroxetine HCL (PAR) will be given as an oral suspension (10 mg/5 mL) per NG/OGtube or PO if the patient is able to swallow. PAR suspension is stored at or below 77° F. The T1/2 of elimination is 21 to 24 hours (50% of drug is eliminated within 21 hours of stopping). Maximum duration of the intervention will be 3 days. All clinically indicated medications are allowed to be given concomitantly. Between 10-100 mg/kg of valproate sodium (VAL) will be given daily as an oral suspension per NG/OG tube or PO if the patient is able to swallow. The drug is stored at 59°−86° F. The T1/2 of elimination is 8-17 hours (so 50% of the drug is eliminated within 8 hours of stopping). Maximal duration of intervention will be 3 days. All clinically indicated medications are allowed to be given concomitantly.

Valproate (VAL) levels are monitored prior to Day 2 and 3 doses. All other interventions are standard. Respiratory and infectious outcomes (using consensus definitions) will be followed as will ventilator-, ICU- and hospital-free days. Pneumonia diagnoses will be based on clinician impression confirmed by bacteriologic studies of semi-quantitative bronchoalveolar lavage (BAL or BALs) when available. Adverse events (AE/SAE) will be recorded. Standard safety end-points will be followed for 12 months post injury. Blood and airway specimens will be obtained on admission and Day 3 (or ICU discharge, whichever is later). Circulating WBC will be isolated from whole blood and cells frozen for CyTOF. Plasma and BAL specimens will undergo 71-plex Luminex assays and infectious diagnoses will be further confirmed by multiplex mediator phenotypic analyses (MMP). Efficacy of VAL+PAR on nosocomial infection will be determined.

What is claimed is:

1. A method of treating a trauma patient, comprising:
(a) administering a HDAC inhibitor to the trauma patient; and
(b) administering a GRK2 inhibitor to the trauma patient.

2. The method of claim 1, wherein the HDAC inhibitor and the GRK2 inhibitor are administered at the same time.

3. The method of claim 1, wherein the HDAC inhibitor and the GRK2 inhibitor are administered sequentially.

4. The method of claim 3, wherein the HDAC inhibitor is administered before the GRK2 inhibitor is administered.

5. The method of claim 3, wherein the HDAC inhibitor is administered after the GRK2 inhibitor is administered.

6. The method of any one of claims 1-5, wherein the HDAC inhibitor comprises vorinostat, panobinostat, belinostat, romidepsin, chidamide, valproic acid, tacedinaline, mocetinostat, abexinostat, practinostat, resminostat, givinostat, quisinostat, HBI-8000, or combinations thereof.

7. The method of claim 6, wherein the HDAC inhibitor is valproic acid.

8. The method of any one of claims 1-7, wherein the GRK2 inhibitor comprises a selective serotonin reuptake inhibitor, GSK180736A, CMPD101, CMPD103, or combinations thereof.

9. The method of claim 8, wherein the GRK2 inhibitor is paroxetine.

10. The method of any one of claims 1-9, wherein the HDAC inhibitor comprises oral administration, intravenous administration, transdermal administration, inhalation administration, or intraosseous vascular administration.

11. The method of any one of claims 1-10, wherein the GRK2 inhibitor comprises oral administration, intravenous administration, transdermal administration, inhalation administration, or intraosseous vascular administration.

12. The method of any one of claims 1-11, wherein the trauma comprises clinical trauma, physical trauma, or combat trauma.

13. The method of claim 12, wherein the clinical trauma comprises surgery, injury, tissue damage, infection, inflammation, pain, medical treatment, secondary disease, or combinations thereof.

14. The method of claim 13, wherein the infection is a nosocomial infection.

15. The method of claim 14, wherein the nosocomial infection is pneumonia.

16. The method of claim 15, wherein the nosocomial infection comprises post-injury pneumonia.

17. A method of treating a nosocomial infection in a patient, the method comprising:
(a) administering a HDAC inhibitor to the subject; and
(b) administering a GRK2 inhibitor to the subject, thereby treating the nosocomial infection in the subject.

18. The method of claim 17, wherein the HDAC inhibitor and the GRK2 inhibitor are administered at the same time.

19. The method of claim 17, wherein the HDAC inhibitor and the GRK2 inhibitor are administered sequentially.

20. The method of claim 19, wherein the HDAC inhibitor is administered before the GRK2 inhibitor is administered.

21. The method of claim 19, wherein the HDAC inhibitor is administered after the GRK2 inhibitor is administered.

22. The method of any one of claims 17-21, wherein the subject has experienced a clinical trauma.

23. The method of claim 22, wherein the HDAC inhibitor is administered after the subject experiences the clinical trauma.

24. The method of claim 22, wherein the GRK2 inhibitor is administered after the subject experiences the clinical trauma.

25. The method of claim 22, wherein the HDAC inhibitor and the GRK2 inhibitor are both administered after the subject experiences the clinical trauma.

26. The method of any one of claims 22-25, wherein the clinical trauma comprises surgery, injury, tissue damage, infection, inflammation, pain, medical treatment, secondary disease, or combinations thereof.

27. The method of claim 26, wherein the infection is a nosocomial infection.

28. The method of claim 27, wherein the nosocomial infection is pneumonia.

29. The method of claim 28, wherein the nosocomial infection comprises post-injury pneumonia.

30. The method of any one of claims 17-29, wherein the HDAC inhibitor comprises vorinostat, panobinostat, belinostat, romidepsin, chidamide, valproic acid, tacedinaline, mocetinostat, abexinostat, practinostat, resminostat, givinostat, quisinostat, HBI-8000, or combinations thereof.

31. The method of claim 30, wherein the HDAC inhibitor is valproic acid.

32. The method of any one of claims 17-31, wherein the GRK2 inhibitor comprises a selective serotonin reuptake inhibitor, GSK180736A, CMPD101, CMPD103, or combinations thereof.

33. The method of claim 32, wherein the GRK2 inhibitor is a selective serotonin reuptake inhibitor.

34. The method of claim 33, wherein the GRK2 inhibitor comprises citalopram, escitalopram, fluoxetine, paroxetine, sertraline, or combinations thereof.

35. The method of claim 34, wherein the GRK2 inhibitor is paroxetine.

36. The method of any one of claims 17-35, wherein the HDAC inhibitor comprises oral administration, intravenous administration, transdermal administration, inhalation administration, or intraosseous vascular administration.

37. The method of any one of claims 17-36, wherein the GRK2 inhibitor comprises oral administration, intravenous administration, transdermal administration, inhalation administration, or intraosseous vascular administration.

38. A method of prophylactically treating a subject, the method comprising:
(a) administering a HDAC inhibitor to the subject; and
(b) administering a GRK2 inhibitor to the subject, wherein the subject is at risk of experiencing trauma.

39. The method of claim 38, wherein the HDAC inhibitor and the GRK2 inhibitor are administered at the same time.

40. The method of claim 38, wherein the HDAC inhibitor and the GRK2 inhibitor are administered sequentially.

41. The method of claim 40, wherein the HDAC inhibitor is administered before the GRK2 inhibitor is administered.

42. The method of claim 40, wherein the HDAC inhibitor is administered after the GRK2 inhibitor is administered.

43. The method of claim 38, wherein the HDAC inhibitor is administered before the subject experiences the trauma.

44. The method of claim 38, wherein the GRK2 inhibitor is administered before the subject experiences the trauma.

45. The method of claim 38, wherein the HDAC inhibitor and the GRK2 inhibitor are both administered before the subject experiences the trauma.

46. The method of claim 38, wherein the HDAC inhibitor is administered after the subject experiences the trauma.

47. The method of claim 38, wherein the GRK2 inhibitor is administered after the subject experiences the trauma.

48. The method of claim 38, wherein the HDAC inhibitor and the GRK2 inhibitor are both administered after the subject experiences the trauma.

49. The method of claim 38, wherein the HDAC inhibitor is administered before and after the subject experiences the trauma.

50. The method of claim 38, wherein the GRK2 inhibitor is administered before after the subject experiences the trauma.

51. The method of claim 38, wherein the HDAC inhibitor and the GRK2 inhibitor are both administered before and after the subject experiences the trauma.

52. The method of any one of claims 38-51, wherein the HDAC inhibitor comprises vorinostat, panobinostat, belinostat, romidepsin, chidamide, valproic acid, tacedinaline, mocetinostat, abexinostat, practinostat, resminostat, givinostat, quisinostat, HBI-8000, or combinations thereof.

53. The method of claim 52, wherein the HDAC inhibitor is valproic acid.

54. The method of any one of claims 38-53, wherein the GRK2 inhibitor comprises a selective serotonin reuptake inhibitor, GSK180736A, CMPD101, CMPD103, or combinations thereof.

55. The method of claim 54, wherein the GRK2 inhibitor is a selective serotonin reuptake inhibitor.

56. The method of claim 55, wherein the GRK2 inhibitor comprises citalopram, escitalopram, fluoxetine, paroxetine, sertraline, or combinations thereof.

57. The method of claim 56, wherein the GRK2 inhibitor is paroxetine.

58. The method of any one of claims 38-57, wherein the HDAC inhibitor comprises oral administration, intravenous administration, transdermal administration, inhalation administration, or intraosseous vascular administration.

59. The method of any one of claims 38-58, wherein the GRK2 inhibitor comprises oral administration, intravenous administration, transdermal administration, inhalation administration, or intraosseous vascular administration.

60. The method of any one of claims 38-59, wherein the trauma comprises surgery, injury, tissue damage, infection, inflammation, pain, medical treatment, secondary disease, or combinations thereof.

61. The method of claim 60, wherein the infection is a nosocomial infection.

62. The method of claim 61, wherein the infection is pneumonia.

63. The method of claim 62, wherein the infection comprises post-injury pneumonia.
